(19) United States
(12) Patent Application Publication
(10) Pub. No.: US 2003/0045458 A1
(43) Pub. Date: Mar. 6, 2003

(54) METHOD AND COMPOSITION FOR TREATING CANCER BY CONVERTING SOLUBLE RADIOACTIVE TOXIC AGENTS INTO INSOLUBLE RADIOACTIVE TOXIC PRECIPITATES VIA THE ACTION OF NON-MAMMALIAN ENZYMES BOUND TO THE NON- ENDOCYTOSING RECEPTORS OF TARGET CELLS

(76) Inventor: SAMUEL ROSE, OAKLAND, CA (US)

Correspondence Address: JOHN Q MCQUILLAN, 125 CRESTWOOD AVENUE, TUCKAHOE, NY, 10707-2208, US

(21) Appl. No.: 08/782,590

(22) Filed: Jan. 13, 1997

(51) Int. Cl. A61K 38/00; C12P 21/06

(52) U.S. Cl. 514/12; 435/68.1

(57) ABSTRACT

A method for the treatment of cancer is disclosed which is capable of directing supra-lethal doses of radiation, called Hot-Spots, virtually exclusively to the cancer. The present invention involves a multi-step therapy process and includes a class of novel chemical agents. In accordance with the present invention, it was discovered that soluble precipitable materials can be made to accumulate as non-digestible precipitates in the extra-cellular fluid in the cancer region as a result of non-mammalian enzyme action. Precipitate accumulation is achieved by the prior administration of a bispecific reagent with a non-mammalian enzyme moiety and a targeting agent capable of binding to non-endocytosing receptors of target cancer cells. A soluble radioactive toxic therapeutic agent is then administered, the soluble toxic therapeutic agent being adapted to be converted by the non-mammalian enzyme moiety of the bound bispecific reagent into a new form which is retained adjacent to the target cancer cells for an extended period of time, thereby generating Hot-Spots which non-selectively kill all cells in the cancer region adjacent to the bispecific reagent.

The invention relates to a method in the general field of immunotherapy, wherein a particular therapeutic effect is sought to be achieved with respect to particular cells or organisms in humans or animals through the use, either directly or indirectly, of soluble radioactive toxic agents which are introduced into the living host The soluble radioactive toxic agents being adapted to be converted into insoluble radioactive toxic precipitates by the action of previously introduced non-mammalian enzymes bound to the non-endocytosing receptors of targeted cells. These radioactive toxic precipitates generate supra-lethal regions of radiation, called Hot-Spots, which are capable of non-selectively killing cells in the targeted region. In particular, the present invention relates to a method of the treatment of cancer.

### BACKGROUND OF THE INVENTION

1. Field of the Invention

A considerable portion of world-wide research efforts in the treatment of cancer is currently devoted to killing cancer cells by means of various cell killing agents. Despite the fact that numerous drugs, radioactive compounds, and the like have been shown to be capable of killing cancer cells, these agents fail to treat cancer successfully because of their inability to circumvent three universally present obstacles: (1) the agents do not kill all the cancer cells because they do not exhibit cytotoxic specificity for all the cancer cells, (2) the agents also kill normal cells because they do not exhibit cytotoxic specificity exclusively for cancer cells, and (3) the agents are not potent enough to kill resistant cancer cells or to overcome the ability of cancer cells to adapt and become resistant to the cell killing agents. An appreciation of these three obstacles is necessary to understand why current treatments fail and to understand the rational and methodology of the proposed invention.

Fifty years of intense research has shown that there is a wide heterogeneity in every characteristic that has been measured in cancer cells. These characteristics include cell size, buoyancy, anaerobic metabolism, enzyme composition, growth rate, gene errors, differential gene expression, chromosome number, and chromosome errors. The heterogeneity is also expressed by the presence of some cancer cells that are super-sensitive and others that are super-resistant to being killed or treated by any therapeutic agent. Within the same tumor population a fraction of cells will be sensitive to a given therapeutic agent and will be killed when that agent is administered, a fraction of cells will be resistant to the agent and will not be killed, and a fraction will adapt and become increasingly more resistant to subsequent therapeutic regimens. The resistant cells will continue to divide and spread to distant locations in the body to form metastatic tumors.

The wide heterogeneity in sensitivity to any particular therapeutic agent leads to the high probability that the systemic application of any therapeutic agent will cause partial remission of the tumor by killing the super-sensitive cancer cells, but will not be able to achieve a complete cure because it cannot kill the super-resistant cancer cells. Previous attempts at cancer therapy have generally ignored the negative therapeutic consequences of these divergent cells. There has been an intuitive and optimistic belief than an approach achieving partial remission in its early phase will give a complete cure after it has been fine-tuned. This optimism contradicts the biological principle, supported by a large amount of data, that every large population of cells or organisms is heterogeneous, and that cancer cells, which have a genetic instability, exhibit a particularly high degree of heterogeneity. Therefore, it is not surprising that the past history of cancer therapy approaches has been a monotonous sequence of short periods of hope, because killing some cancer cells leads to a remission, followed by prolonged periods of disappointment, because some cancer cells survive, seed, and continue to grow in the living host and subsequent treatments are less effective at killing the cancer cells of these metastatic tumors. It is likely that the latest field of oncogenes and other gene manipulations, as applied to cancer therapy, will also follow the same pattern. This prediction is based on the fact that there is a heterogeneity of gene errors and gene expression in the cancer cell population, and with time, more and more cells, with more and varied genetic and chromosome errors accumulate in the cancer cell population. No simple genetic correction, even if it could be applied successfully to all the cancer cells growing in the body, is likely to repair every cell.

2. Prior Art

The first serious deficiency of current cancer therapeutic approaches is that they do not take into account, and are unable to deal with, the heterogeneity of the cancer cell population. The inability of current approaches to circumvent this heterogeneity is illustrated by the failure of immuno-therapeutic approaches that rely on antigenic receptors on the surface of cancer cells to deliver therapeutic agents.

All current attempts at cancer therapy (apart from the treatment of thyroid cancer with radio-iodide) depend on killing each and every individual cancer cell by their direct individual interaction with the candidate therapeutic agents or applied environmental condition. In order to describe the need for this direct interaction, these strategies can be loosely called “sniper killing,” i.e. each cell to be killed must be targeted directly. Sniper killing agents include cytotoxic drugs, binary reagents made by attaching cancer targeting agents to cytotoxic drugs, augmented immune response, hormonal therapy, genetically engineered products (like interferon), manipulations of oncogenes, or products coded by these genes.

In order for these sniper killing strategies to be successful in treating cancer, it would be necessary for the cancer cells to have an exploitable characteristic which is present on all cancer cells, for that characteristic to be absent from all (or at least most) normal cells, and for that characteristic to not adaptively change and become non-exploitable.

It is known that cancer cells exhibit on their surface numerous receptors, including antigenic receptors, to which selected molecules such as specific antibodies, hormones, and peptides can bind. Antibodies, hormones, and peptides can be used as targeting agents for the cancer cells that express those particular antigenic receptors. Ideally, all cancer cells would express the receptor, and the number of non-cancerous cells which express the receptor would be very small. In the ideal model, binary reagents (an example of a sniper strategy) which are composed of targeting agents and cytotoxic agents would be preferentially directed to the cancer cells. However, in practice, binary reagents do not result in the delivery of the cytotoxic agent to all cancer cells in the tumor population because some cancer cells do not exhibit the particular antigenic receptor. The binary reagent will not attach to these antigenic receptor deficient cancer cells, and therefore these cells will be unaffected by the treatment and will be left to proliferate in the host. High-dose sniper killing, even when employed at dose levels which kill many normal cells, fail to kill all cancer cells because some cancer cells are antigenic receptor deficient, some cancer cells are super-resistant even before the treatment begins, and some cancer cells adapt to the therapeutic agent, survive, and become resistant to future treatments. All these sniper strategies have failed, and are doomed to fail in the future, because they cannot deal with the fact that some normal cells also express the characteristic which is the target for the sniper killing, and because they cannot deal with the universally present heterogeneity and adaptive ability of cancer cells.

The recent development of highly pure and highly immuno-specific monoclonal antibodies. hormones, and peptides which can act as specific targeting agents for particular antigenic receptors has greatly increased the ability to direct cell killing agents specifically to cancer cells and thereby minimize any adverse effects on non-cancerous cells. Paradoxically, this current direction of isolating and producing such highly specific targeting agents (for the purpose of minimizing the possibility that such antibodies, and the cytotoxic agents carried thereby, might attach to non-cancerous cells) is, in one sense, counter-productive, since the number of cancer cells within the tumor population which will exhibit an affinity for such highly specific targeting agents will be reduced.

Notwithstanding the above-mentioned advances in the development of highly specific targeting agents to deliver the cell killing agents specifically to targeted cells, and the demonstrated cell killing ability of the particular delivered agents, therapeutic success through the use of binary reagents composed of targeting agents and toxic agents has not been achieved, and should not have been expected. Unfortunately, in practice these therapies have been far less successful than they were hoped to be.

The second serious deficiency of binary reagents to carry cytotoxic agents to target cancer cells is that the so called “cancer targeting agents” of which the binary reagents are made, also target a significant number of normal cells. These targeted normal cells are also killed by the administration of binary reagents, cause unacceptable destruction of normal tissues, serious illness of the patient, and limit the aggressiveness of the attack which can be launched against the cancer.

The third serious deficiency of binary reagents to carry cytotoxic agents to target cancer cells, particularly cytotoxic radioactive isotopes, is that they cause significant systemic toxicity because the targeting agent carrying the cytotoxic agent is a large molecule which causes them to have a long residence time in the blood circulation, and causes them to be taken up non-specifically by normal cells.

The fourth serious deficiency of binary reagents to carry cytotoxic agents to cancer cells is that even those cancer cells which the targeting agents attaches to, outright killing of the cancer cell is often not accomplished. In large part this is due to the inherent limitations of the treatment method, i.e., the absolute quantity of cytotoxic agent which can be coupled to the targeting agent is smaller than that required to actually kill the cancer cell (the small quantity of cytotoxic agent which can be attached is limited to avoid destroying the targeting ability of the targeting agent and to avoid adversely altering the distribution of the binary reagent in the host). While the amount of cytotoxic agent which can be brought to bear on cancer cells through the use of binary reagents may be sufficient to damage some of the cells, the damage often is only temporary or, indeed, simply results in the emergence of mutant cells which are still cancerous and have become resistant to the effects of the cyrotoxic agent.

The fifth serious deficiency of the binary reagents to carry cytotoxic agents is that it is impossible to make a valid choice of the most appropriate targeting agent to make the binary reagent for each cancer in each patient. Furthermore, it is not possible to predict the outcome of the therapy prior to administering the binary reagent at the necessary cytotoxic dose level.

Despite the three obstacles and the deficiencies described above, the treatment of thyroid cancer with radio-iodide is successful in a high proportion of cases. This high rate of success is not due to a fundamental difference between cancer cells of the thyroid and cancer cells which have arisen from other tissues. The successful treatment of thyroid cancer is due to the fact that normal and malignant thyroid cells have a unique biological function which allows them to store iodine. Thus, when patients with thyroid cancer are treated with radio-iodide, a fraction of the cancer cells take up sufficient quantities of isotope and store the isotope long enough to generate micro-regions of intense radiation in which all the cells in each micro-region are killed. These intense radiation fields, called Hot-Spots, are generated exclusively in the normal and malignant thyroid tissue. The radiation field in the Hot-Spots extends beyond the cells taking up the isotope and kills hundreds of neighboring cells thereby creating overlapping micro-regions of supra-lethal radiation (overlapping Hot-Spots) exclusively in the thyroid tissue. Inside these Hot-Spots, the radiation is so intense that all the cancer cells in the tumor are killed, including the cells that do not take up the radio-isotope.

Two types of strategies have been employed to amplify and localize the effect of cytotoxic agents on targeted cells in order to circumvent the five deficiencies described and in order to simulate the operating conditions that make the treatment of thyroid cancer so successful. The first strategy is to form the cytotoxic agents on the surface of the targeted cells, and the second strategy attempts to accumulate the cytotoxic agents outside cells in the extra-cellular fluid.

The first strategy to amplify and localize the effect of cytotoxic agents on targeted cells is to accumulate agents on the surface of targeted cells. Accumulation of radioactive toxic materials on the surface of targeted cells has been achieved by a single step direct delivery of radio-labeled targeting agent which can bind directly to the non-endocytosing receptors of targeted cells. The single step method consists of the administration of a radio-labeled protein or peptide targeting agent that can directly bind to the targeted non-endocytosing cell receptors. In order to overcome the problem of systemic toxicity caused by the long residence time of the targeting agent in the circulating blood (the long residence time being caused by the slow diffusion out of the blood capillaries of the targeting agent which is a large molecule) a three step indirect delivery of radio-isotopes to the non-endocytosing receptors of targeted cells has been used. In the three step method, the isotope carrying molecule is small enough to circulate rapidly throughout the body fluids and thus have a short residence time in the blood. The three-step, indirect delivery method consists of: (i) administering a bispecific reagent which will bind to the non-endocytosing target cell receptors, the bispecific reagent comprising an antibody having a substantial affinity for the non-endocytosing receptors on cancer cells and further having an antibody or peptide which has a substantial affinity for, and can bind to, the small radio-isotope carrying molecules; (ii) waiting for a period of time to allow for the elimination from the body of bispecific reagent not specifically bound to the non-endocytosing receptors on targeted cancer cell receptors; and (iii) after the elimination of all bispecific reagent, not specifically bound to the targeted cell receptor is completed, administering a small isotope carrying molecule. The small isotope carrying molecule circulates in the blood, diffuses rapidly into the extra-cellular fluid, and binds to the non-endocytosing receptors via the bound bispecific reagent (Corvalan et al., 1987 Cancer immunology and immunotherapy, 24,133-137; Corvalan et al., 1988, Int J. of Cancer, 2 Supple. 2-25; Glennie et al. 1988, J. of Immunology, 141, 3662-3670; Stickney et al., 1989, In Fourth Int Conf. on Monoclonal Antibody Immunoconjugate For Cancer, p 29, San Diego,-UCSD).

The second strategy to amplify and localize the effect of cytotoxic agents on targeted cells is to form the cytotoxic agents outside targeted cells in the extra-cellular fluid. The formation of cytotoxic agents outside targeted cells in the extra-celluar fluid of the targeted regions has been achieved by the enzymatic conversion of a pro-drug into an active drug by a method called Antibody Dependent Enzyme Pro-Drug Therapy (ADEPT). The enzyme which makes the conversion is one moiety of a bispecific reagent, the other moiety being an antibody with a binding affinity to the non-endocytosing receptors on surface of targeted cancer cells. Since the enzyme moiety is bound to the surface of the targeted cells, the conversion from pro-drug to the active drug takes place in the extra-cellular fluid.

The active drug diffuses into the immediate micro-region to have its pharmacological cytotoxic effect on the non-target cancer cells in the raicro-region. For example, alkaline phosphatase converts the pro-drugs mitomycin phosphate into an active mitomycin C derivative and etoposide phosphate into an active etoposide (Senter et al, 1989, Cancer Research, 49, 5789-5792), beta-lactamase converts a cephalosporin derivative of 4-de-succetylvinblastine-3-carboxhydrazide into an active cytotoxic drug (Meyer et al, 1993, Cancer research, 53, 3956-3963), and activates cephalo-doxorubcin (Rodrigues et al, 1995, Cancer Research, 55, 63-70), DT diaphorase followed by a non-enzymatic reaction with a thioester activates the mono-functional alkylating agent CB 1954 into an active agent which can cause ctotoxicity by cross-linking DNA (Knox et al, 1993, Cancer and Metastasis Reviews, 12, 195-212); carboxypeptidase G2 can convert a nitrogen mustard pro-drug into an active drug (Springer and Niculescu-Duvaz, 1995, Anticancer Drug Des. 10, 361-372); nitroreductase can activate CB 1954 (Knox et al. 1995, Biochem. Pharmacol., 49, 1641-1647); and dinitrobenzamide (Anlezark et al, 1995, Biochem. Pharmacol., 50, 609-618); to form cytotoxic derivatives, and alpha-galactosidase can activate prodrugs of anthracycline (Azoulay et al, 1995, Anticancer Drug Des., 10, 441-450).

The three step ADEPT approach fails to successfully treat cancer for the following reasons: (a) the bispecific reagent is bound to the non-endocytosing target cancer cells and also to some normal cells because the targeting agent moiety does not exhibit exclusive cytotoxic specificity for cancer cells which reduces the tumor specificity of the non-mammalian enzyme location and pro-drug conversion, (b) the antigenic receptors of the target cells are in a constant state of flux which prevents the bispecific reagent from remaining bound for a sufficient period of time to allow all bispecific reagent not bound specifically to the target cell receptors to be eliminated from the body prior to administering the pro-drug; (c) the soluble active drug which is made by the enzyme diffuses away from its site of production to have a cytotoxic action on healthy normal cells; (d) the cells on which the bispecific reagent is bound, and where the active drug is formed, are the first cells to be killed because they receive the highest concentration of the active drug. When these cells are killed, the enzyme will no longer be in a position to convert the pro-drug into an active drug and, therefore, the production of active drug is self limiting; and (e) the shape and volume of the micro-region in which there is a sufficiently high concentration of the active drug to kill cells is variable and ill-defined because the diffusion parameters of the soluble active drug are dependent on the particular status of the blood capillaries and extra-cellular structures in the cancer, the parameters of the diffusion varying from one location of the tumor to another.

The two strategies described above fail to generate Hot-Spots because the number of cytotoxic chemical or radio-isotope agents which are delivered is small, the number being directly proportional to the relatively small number of antigenic receptors on the surface of the target cells. In addition, the agents or isotopes do not remain in the correct location for long enough to achieve an aggressive attack on the cancer, and furthermore, they cause systemic toxicity because the agents circulate in the blood for a long period of time. Finally these strategies also fail to locate the attack specifically to the tumor, because the location where the agent or isotopes are delivered or where the active drug is made is dependent on only a single cancer associated characteristic on the cancer cell surface, and every single characteristic found on cancer cells is also found on some normal cells.

The present invention mimics for non-thyroid cancers, the Hot-Spot killing which makes the treatment of thyroid cancer successful; however, since no other malignant tissue has the same natural iodide involving process as the thyroid, the mimicking requires the construction of a special, multi-step, sequential process to achieve “Hot-Spots” in non-thyroid cancers. The basic process of the present invention consists of sequential steps which act independently and together with naturally occurring characteristics of the cancer and normal cell populations to generate overlapping Hot-Spots virtually exclusively in the tumor without causing significant systemic toxicity. Cancer cells within these Hot-Spots are eradicated, the eradicated cells include cancer cells that are not targeted, cancer cells that are resistant and even super-resistant, and cancer cells that would otherwise adapt and become resistant to therapy.

### SUMMARY OF INVENTION

An object of the present invention is to provide a method for the treatment of cancer that directs supra-lethal doses of radiation in the form of micro-regions of radiation, called Hot-Spots, virtually exclusively to the cancer. All the cells, numbering in the thousands, within each Hot-Spot are killed, therefore, the method of the present invention does not require each individual cancer cell to be targeted in order for every cancer cell in the living host to be killed. As a consequence the method of the present invention is not defeated by the heterogeneity of cancer cell receptors and the imperfect nature of current targeting agents. Because the Hot-Spots are located virtually exclusively in the region of the cancer, the present invention does not kill healthy cells and will not cause significant systemic toxicity.

It is another object of the present invention to provide a method for accumulating at the non-endocytosing receptors of target cells non-mammalian enzymes capable of converting a soluble therapeutic agent into an insoluble precipitate. The non-mammalian enzyme is accumulated via the administration of bispecific reagents comprised of a non-mammalian enzyme moiety and a targeting agent moiety with a substantial affinity for the non-endocosing receptors of target cancer cells.

It is a further object of the present invention to accumulate non-toxic and toxic therapeutic precipitates in the extra-cellular fluid of the cancer region. Precipitate are accumulated via the administration of a soluble precipitable material which is adapted to be converted into an insoluble precipitate by the non-mammalian enzyme moiety of the bispecific reagent.

It is still a further object of the present invention to provide a method for the immunological treatment of any cell population or organism for therapeutic purposes.

It is an additional object of the present invention to provide a bispecific reagent adapted to have a non-mammalian enzyme moiety and a targeting agent moiety, the bispecific reagent being adapted to bind to the non-endocytosing receptors of targeted cells. The bispecific reagent having a targeting agent moiety adapted to have a substantial affinity for the non-endocytosing receptors on target cancer cells the enzyme moiety being adapted to convert a soluble therapeutic agent into an insoluble precipitate.

### BRIEF DESCRIPTION OF THE DRAWINGS

FIG. 1 shows the first target cancer cells.

FIG. 2 shows the second target cancer cells.

FIG. 3 shows the third target cancer cells.

FIG. 4 shows the first target normal cells.

FIG. 5 shows the first bispecific reagent.

FIG. 6 shows the first bispecific reagent binding to the first target normal cells.

FIG. 7 shows the first bispecific reagent binding to the first target normal cells.

FIG. 8 shows the first therapeutic agent being converted by the non-mammalian enzyme of the first bispecific reagent into the first extra-cellular precipitate.

FIG. 9 shows the dimerization of the first therapeutic agent.

FIG. 10 shows the second extra-cellular precipitate being indigo dye.

FIG. 11 shows the method of attaching penicillin to indoxyl phosphate at position 3 via phosphate group.

FIG. 12 shows penicillin attached to indoxyl phosphate at position 3 via phosphate group.

FIG. 13 shows the liberation of indoxyl phosphate from A-B-C and its precipitation by phosphatase.

FIG. 14 shows the attachment of penicillin directly to indoxyl which is then treated; beta lactamase liberates indoxyl which forms a precipitate.

FIG. 15 shows the method of radio-iodinating indoxyl compounds to make iodinated (parahydroxy-benzyl ether of 5-hydroxy indoxyl phosphate).

FIG. 16 shows the method of radio-iodinating indoxyl compounds to make iodinated (parahydroxy-phenyl) substituted at the 5 position.

FIG. 17 shows the method of radio-iodinating indoxyl compounds where 1-acetyl-5 iodo-3-hydroxyindole is exchanged with radioactive iodine.

FIG. 18 shows the precipitation of a soluble precipitable material where the soluble moiety is cleaved from the insoluble moiety by beta lactamase causing the insoluble moiety to precipitate spontaneously.

FIG. 19 shows the accumulation of the first extra-cellular precipitate forming adjacent to the first bispecific reagent.

FIG. 20 shows the second bispecific reagent having the second enzyme moiety and a targeting agent moiety capable of binding to the first extra-cellular precipitate.

FIG. 21 shows the bispecific reagent binding to the first antigenic epitope of the first extra-cellular precipitate.

FIG. 22 shows the bispecific reagent binding to the second antigenic epitope of the first extra-cellular precipitate.

FIG. 23 shows the bispecific reagent binding to the neo-antigenic third epitope of the first extra-cellular precipitate.

FIG. 24 shows the second therapeutic agent being converted by the second enzyme moiety of the second bispecific reagent into the second extra-cellular precipitate.

FIG. 25 shows the dimerization of the second therapeutic agent.

FIG. 26 shows the second extra-cellular precipitate being a radioactive toxic indigo dye.

FIG. 27 shows the method of attaching penicillin to indoxyl phosphate at position 3 via phosphate group.

FIG. 28 shows penicillin attached to indoxyl phosphate at position 3 via phosphate group.

FIG. 29 shows the liberation of indoxyl phosphate from A-B-C and its precipitation by phosphatase.

FIG. 30 shows the attachment of penicillin directly to indoxyl which is then treated; beta lactamase liberates indoxyl which forms a precipitate.

FIG. 31 shows the precipitation of a soluble precipitable material where the soluble moiety is cleaved from the insoluble moiety by beta lactamase causing the insoluble moiety to precipitate spontaneously.

FIG. 32 shows the third therapeutic agent being converted by the non-mammalian enzyme of the bispecific reagent into the new form of the third therapeutic agent.

FIG. 33 shows the third therapeutic agent being chondroitin sulphate being converted by chondroitinase into a new form.

FIG. 34 shows the conversion of the third therapeutic agent into a new form which is soluble which is then acted on by a precipitating antibody to form the third extra-cellular precipitate.

FIG. 35 shows the third bispecific reagent tethering the first extra-cellular precipitate to the second target cancer cells.

FIG. 36 shows the fourth bispecific reagent tethering the first extra-cellular precipitate to the cancer-altered extra-cellular matrix.

FIG. 37 shows the fifth bispecific reagent tethering the first extra-cellular precipitate to the relocated natural intra-cellular contents of cells.

FIG. 38 shows the sixth bispecific reagent tethering the second extra-cellular precipitate to the second target cancer cells.

FIG. 39 shows the seventh bispecific reagent tethering the second extra-cellular precipitate to the cancer-altered extra-cellular matrix.

FIG. 40 shows the eighth bispecific reagent tethering the second extra-celluar precipitate to the relocated natural intra-cellular contents of cells.

FIG. 41 shows the ninth bispecific reagent tethering the new form of the third therapeutic agent, which is soluble, to the second target cancer cells.

FIG. 42 shows the tenth bispecific reagent tethering the new form of the third therapeutic agent, which is soluble, to the cancer-altered extra-cellular matrix.

FIG. 43 shows the eleventh bispecific reagent tethering the new form of the third therapeutic agent, which is soluble, to the relocated natural intra-cellular contents of cells.

FIG. 44 shows the cell killing process killing the third target cancer cells and releasing the natural intra-cellular contents of the cells into the extra-cellular fluid.

### DESCRIPTION OF THE PREFERRED EMBODIMENTS

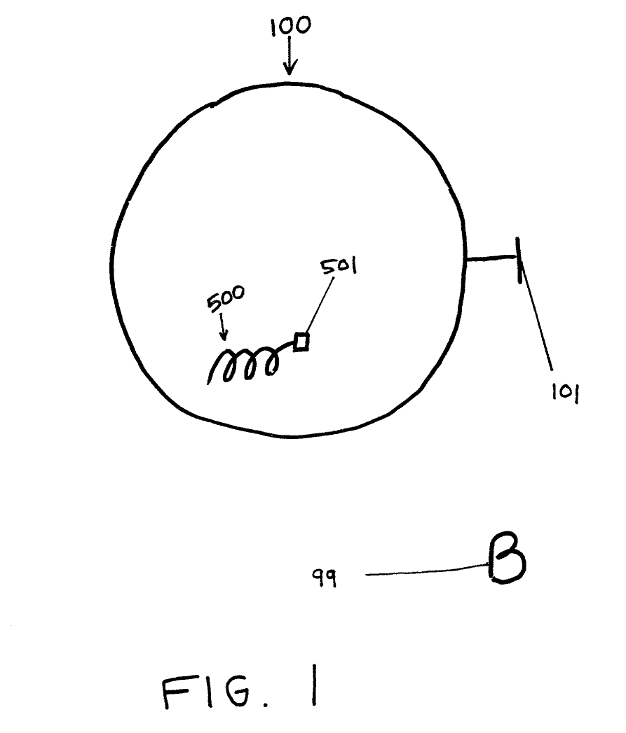

The present invention is a method for treating a heterogeneous population of cancer cells in a living host by at least one of a first therapeutic agent and a second therapeutic agent. The living host being composed of normal cells growing in a normal extra-cellular matrix, the normal extra-cellular matrix having at least collagen and fibronectin, the heterogeneous population of cancer cells growing in a cancer-altered extra-cellular matrix having at least cancer-altered antigenic epitopes 99 (FIG. 1), the heterogeneous population of cancer cells endogenously making and containing products including at least sulphated glycosamninoglycans, natural intra-cellular enzymes in the lysosomes, and natural intra-cellular material including DNA, histone, and complexes of DNA-histone, the DNA, histone and complexes of DNA-histone 500 having antigenic epitopes 501(Epstein et al., 1995, Cancer Research 55, 2673-2680; Akaogi et al., 1996, Proc. Natl. Acad. Sci., 93, 8384-8389). The heterogeneous population of cancer cells include at least three sub-populations of cancer cells.

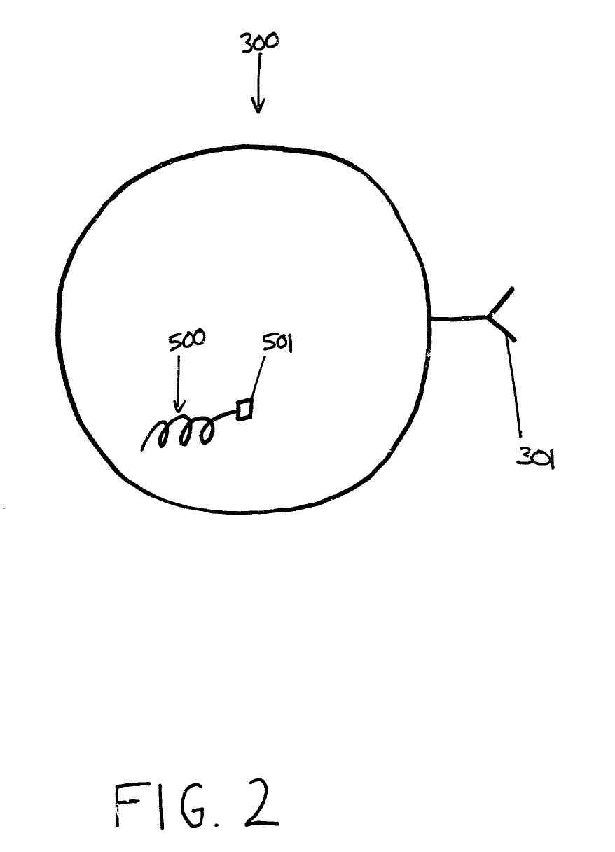

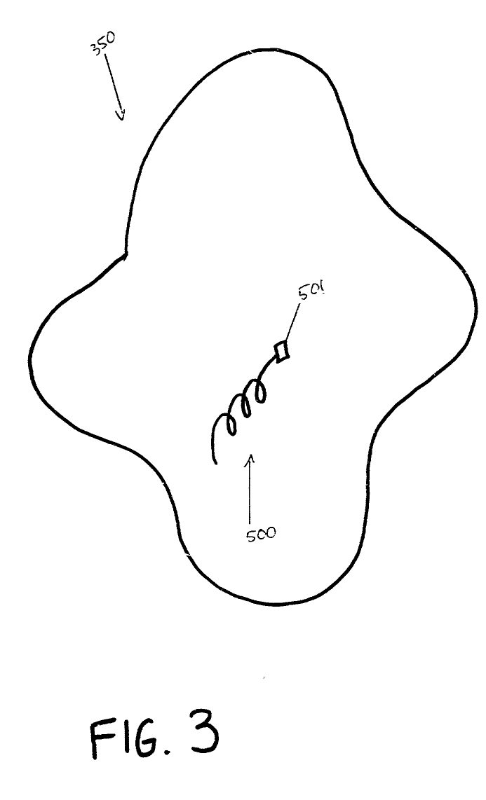

As shown in FIG. 1 the first sub-population of cancer cells being the first target cancer cells 100 each having a first antigenic receptor 101 which is substantially specific to a cancer cell and which is capable of binding a first targeting agent, the first antigenic receptor 101 being incapable of endocytosis when the first targeting agent binds to the first antigenic receptor. The second sub-population of cancer cells, as shown in FIG. 2, being the second target cancer cells 300 each having a third antigenic receptor 301 which is substantially specific to a cancer cell and which is capable of binding a third targeting agent, the third antigenic receptor 301 being incapable of endocytosis. The third sub-population of cancer cells (FIG. 3), being the third target cancer cells 350 each having a high sensitivity to being killed by the natural system of the living host and a high sensitivity to being killed by the cell killing process. The third target cancer cells 350 endogenously making and containing products including at least sulphated glycosaminoglycans, natural intra-cellular enzymes in the lysosomes, and natural intra-cellular material including DNA, histone, and complexes of DNA-histone, the DNA, histone, and complexes of DNA-histone 500 having antigenic epitopes 501. The fourth subpopulation of cancer cells being non-target cancer cells which are the remainder of the cancer cells.

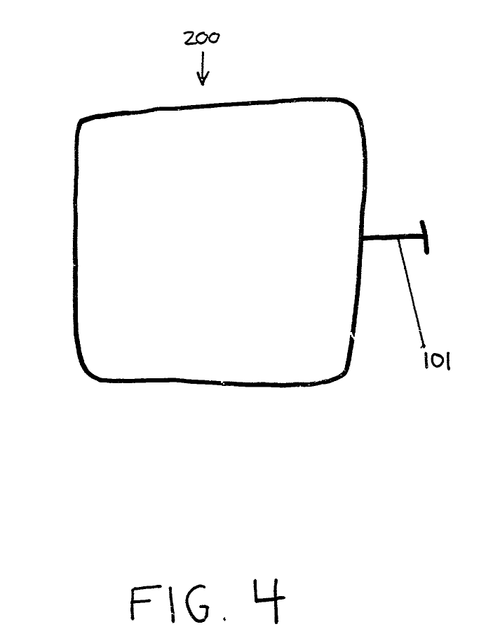

The normal cells of the living host endogenously making and containing products including at least sulphated glycosaminoglycans, natural intra-cellular enzymes in the lysosomes, and natural intra-cellular material including DNA, histone, and complexes of DNA-histone, the DNA, histone, and complexes of DNA-histone having antigenic epitopes. The normal cells including at least two sub-populations of normal cells. As shown in FIG. 4, a first sub-population of normal cells being the first target normal cells 200 which also have the first antigenic receptor 101 which is capable of binding the first targeting agent, the first antigenic receptor being incapable of endocytosis when the first targeting agent binds to the first antigenic receptor. The second sub-population of normal cells being non-target normal cells which are the remainder of the normal cells.

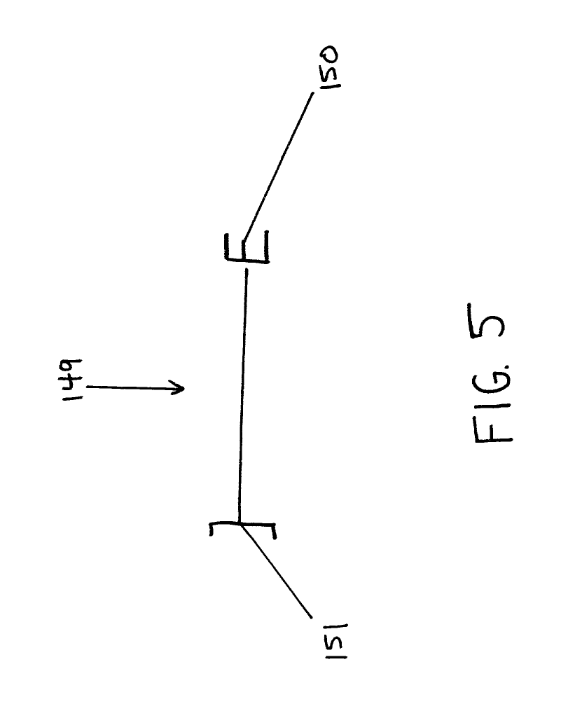

The method of the invention comprising a plurality of steps including at least introducing into the living host a first bispecific reagent. FIG. 5 shows the first bispecific reagent 149 which is introduced to the living host, the bispecific reagent having two moieties, the first moiety being a non-mammalian enzyme moiety being the first enzyme moiety 150. The second moiety of the first bispecific reagent 149 including a targeting agent moiety being the first targeting agent 151. The first targeting agent 151 having a substantial affinity for the first antigenic receptors of the first target cancer cells and the first target normal cells. The first enzyme moiety being adapted to convert a soluble precipitable material being the first therapeutic agent into an insoluble precipitate which is the first extra-cellular precipitate in the extra-cellular fluid adjacent to the first bispecific reagent.

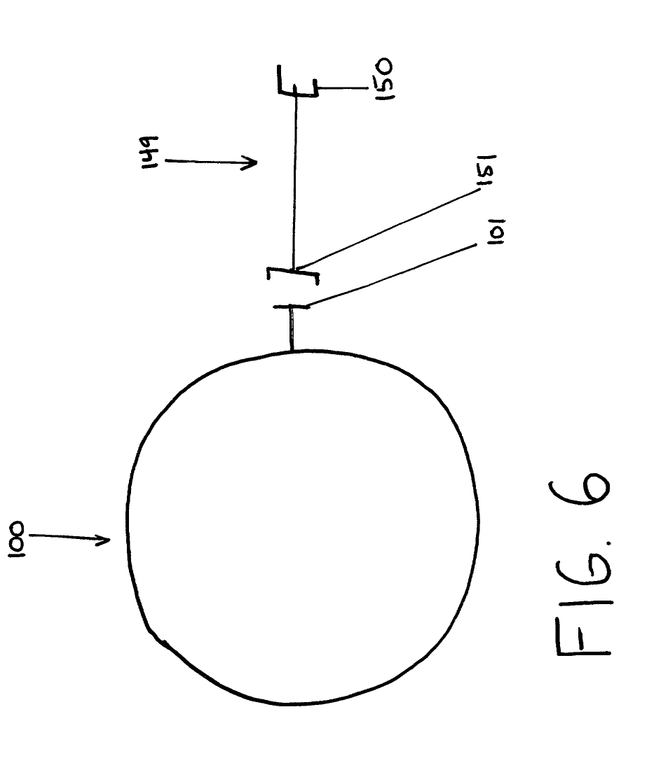

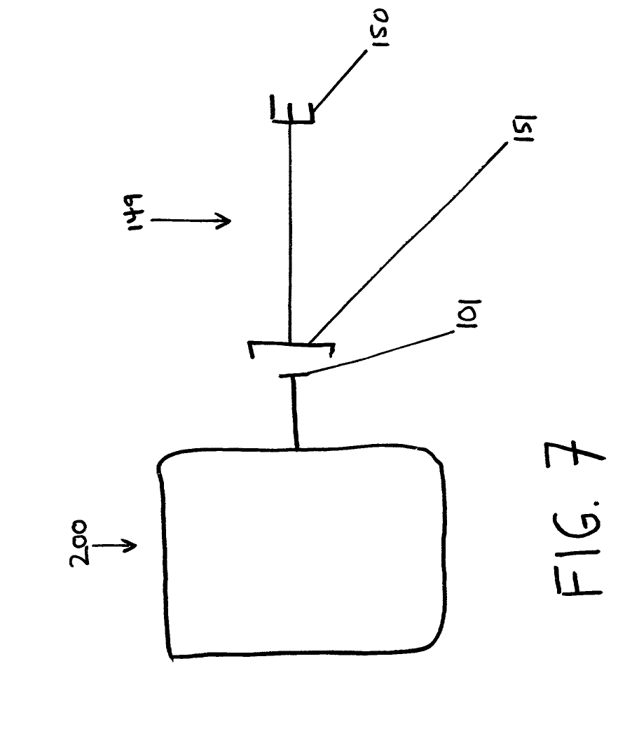

As shown in FIG. 6 the first targeting agent 151 of the first bispecific reagent 149 attaches to the first antigenic receptor 101 of the first target cancer cells 100, thereby retaining the first enzyme moiety 150 the first bispecific reagent 149 in the extrallular fluid. FIG. 7 shows the attachment of the first targeting agent 151 of the first bispecific rent 149 to the first antigenic receptors 101 of the first target normal cells 200, thereby retaining the first enzyme moiety 150 the first bispecific reagent 149 in the extra-cellular fluid.

In accordance with the method of the present invention, after all unbound first bispecific reagent has been eliminated from the living host, the first therapeutic agent, which is a soluble precipitable agent, is administered to the living host. The fist therapeutic agent adapted to be converted in the extra-cellular fluid adjacent to the first bispecific reagent by the first enzyme moiety of the first bispecific reagent into an insoluble non-gestible precipitate which is the first extra-cellular precipitate. The first extra-cellular precipitate being capable of remaining in the extra-cellular fluid adjacent to the first bispecific reagent for an extended period of time.

Since the enzymatic conversion of the fist therapeutic agent takes place exclusively in the extra-cellular fluid, there is no need for the first therapeutic agent to gain entrance into the intra-cellular compartment of the body. Entrance of the first therapeutic agent into cells throughout the living host would increase the volume of distribution of the first therapeutic agent (the intra-cellular compartment has a volume at least 2-3 times that of the extra-cellular fluid) and would increase the duration of time that the first therapeutic agent would circulate in the living host, both these factors—the volume of distribution and the long circulation time—could increase the systemic toxicity caused by the first therapeutic agent. For these reasons it is of significant advantage for the first therapeutic agent to be cell impermeant by being a molecule larger than 1000 daltons and/or being anionic. Alternatively the first therapeutic agent can be made cell impermeant by attaching one of a number of cell impermeant molecules at least including peptides or polymers having a molecular size greater than 1,000 daltons and anionic chemicals including thiols.

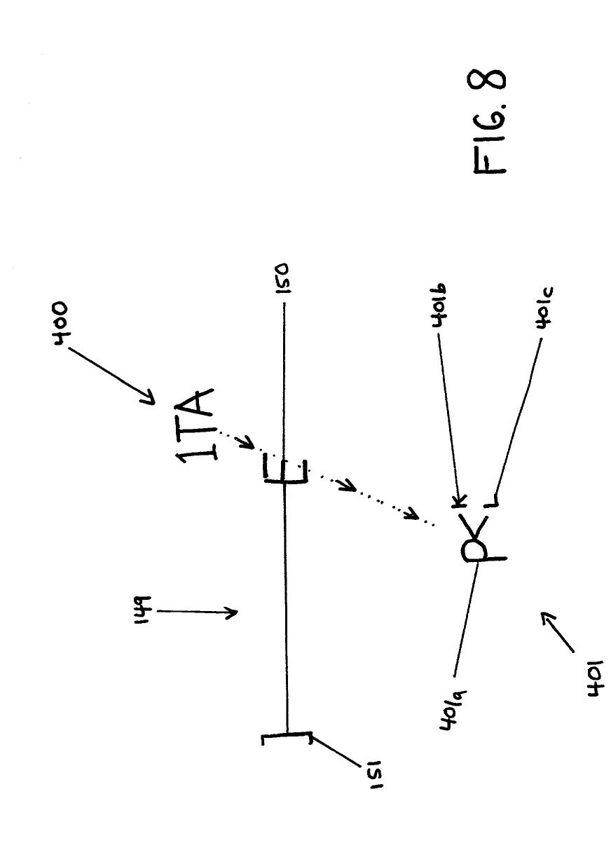

As shown in FIG. 8, the first therapeutic agent 400 is converted by the first enzyme moiety 150 of the first bispecific reagent 149 into an insoluble nongestible precipitate which is the first extra-cellular precipitate 401. The first extra-cellular precipitate having at least one of a first antigenic epitope 401a which is an integral part of the structure of the first extra-cellular precipitate, a second antigenic epitope 401b, and a neoantigenic third epitope 401c, the first antigenic epitope, the second antigenic epitope, and the neo-antigenic third epitope enabling the first extra-cellular precipitate 401 to be “tethered” via previously administered bispecific reagents bound to stable structures in the tumor tissue (as described later). The tethering retaining the first extra-cellular precipitate for an extended period of time in the extra-cellular fluid adjacent to the first bispecific reagent. By retaining the first extra-cellular precipitate in the extra-cellular fluid adjacent to the first bispecific reagent, the first extra-cellular precipitate can act as a platform from which to generate Hot-Spots via the subsequent introducing of the second bispecific reagent and the additional administration of the additional therapeutic agent. The method of generating Hot-Spots is described later.

The first therapeutic agent can be a soluble precipitable agent made by converting chemical X to a soluble XY. The bond attaching X to Y is cleaved by the first enzyme moiety of the first bispecific reagent to create the highly reactive intermediate molecule Xa. The Xa molecule is readily and extremely rapidly oxidized to form Xb. In the oxidized form, Xb spontaneously and covalently selfcondenses or dimerizes to create a new molecule which is insoluble and immediately and spontaneously forms a precipitate. Because a new molecule is formed by the dimerization, the core structure of the precipitate has a neo-antigenic epitope which is not present on the original XY, Xa, Xb or anywhere else in the body.

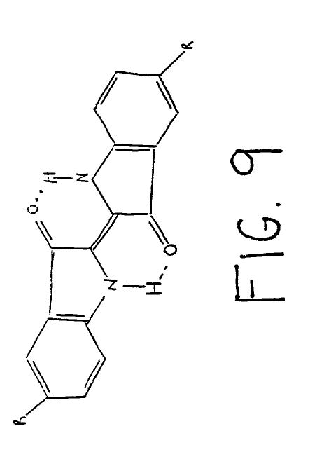

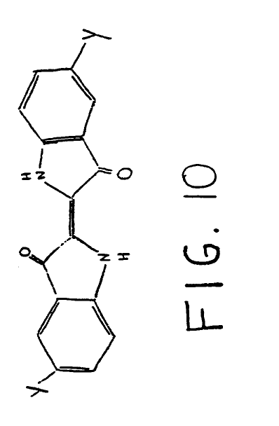

A specific example of a first therapeutic agent to be used is the application of a indoxyl-lactam as examples of XY, which is soluble and can be administered to the living host as a free molecule. The lactam (Y) of the indoxyl-lactam (XY) is cleaved by beta-lactamase engine (being the first enzyme moiety of the first bispecific reagent) to liberate a highly reactive intermediate indoxyl (Xa). As shown in FIG. 9 the indoxyl (Xa) is readily and extremely rapidly oxidized, and once in the oxidized form it spontaneously self-condenses or dimerizes to form a new molecule which is insoluble and precipitates spontaneously as indigo dye as illustrated in FIG. 10 where Y can be aryl, halogen, hydoxyl, and alkyl. The new molecule is different from the indoxyl-lactam and the intermediate indoxyl molecules and thereby having a neo-antigenic epitope not present on the indoxyl-lactam or the indoxyl intermediate.

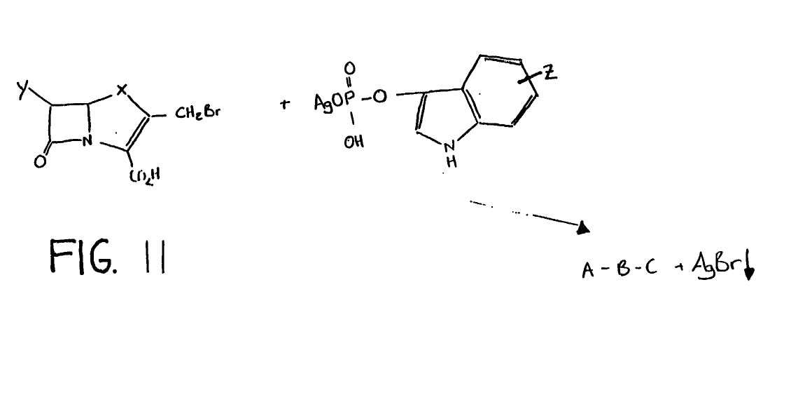

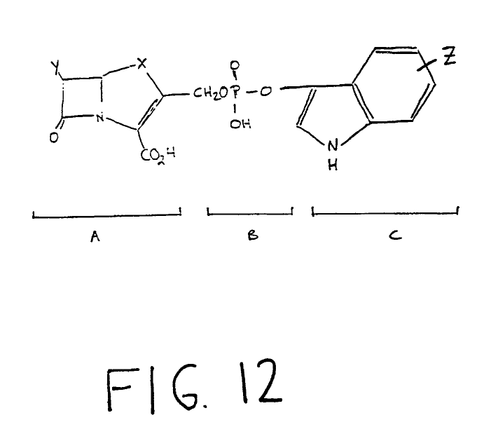

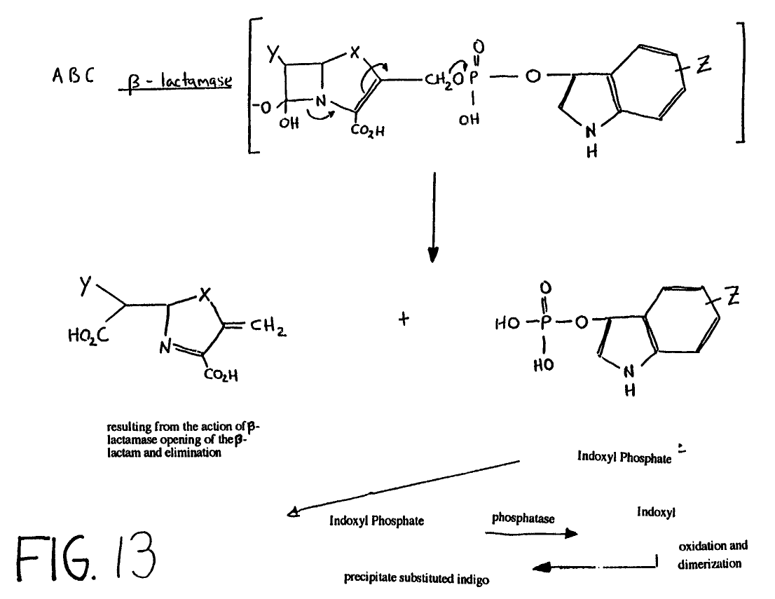

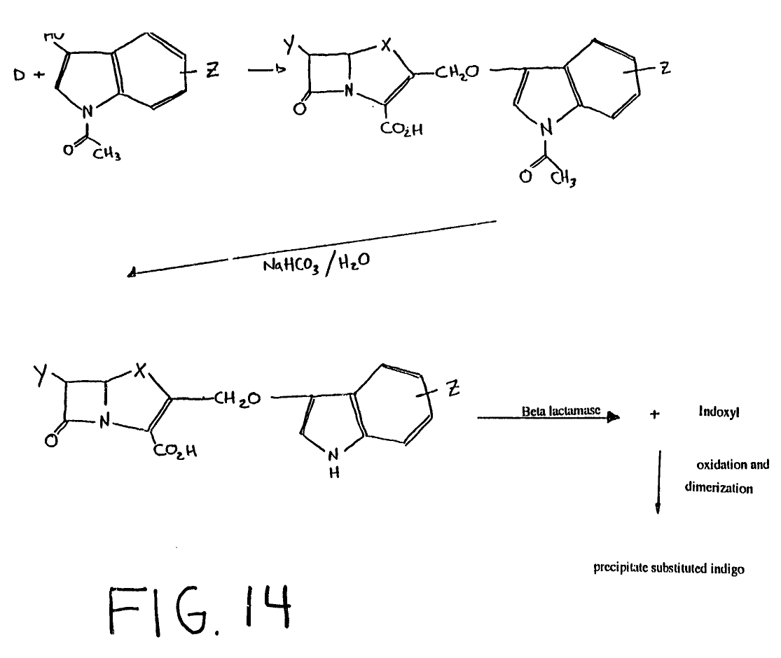

FIG. 11 and FIG. 12 where Y is a typical penicillin at the 6 position, where X is oxygen, sulfur, or carbon, where Z is an appropriate substitute in the indoxyl which allows attachment to targeting agent, alternatively attachment to the targeting agent may be effected through Y, where B is the phosphate (as will be shown similar events may occur with or without the phosphate group), and where C is the substituted indoxyl portion that, when liberated, dimerizes and precipitates. As shown in these diagrams, the lactam can be attached to position 3 of the indoxylphosphate via the phosphate group, in which case it is detached by beta-lactamase to form indoxylphosphate. As shown in FIG. 13 the phosphate group of the remaining indoxylphosphate is cleaved by phosphatase naturally present in body fluids to produce indoxyl which passes through the steps previously described to precipitate. FIG. 14 illustrates how the lactam can also be attached directly to the indoxyl at position 3, in which case precipitation occurs by the direct action of beta-lactaniase.

The oxidation and dimerization of indoxyl proceeds at a slower rate in the acidic pH which is often present in the extra-cellular fluid of the tumor tissue, compared to the rate of oxidation and dimerization in the relatively neutral pH found in the extra-cellular fluid of normal tissues. The slower rate of oxidation and dimerization may allow some of the soluble indoxyl molecules and intermediates to diffuse away from the first enzyme moiety prior to the indoxyl oxidizing, dimerizing, and precipitating. A controlled diffusion away from the first enzyme would have the advantage of distributing the first extra-cellular precipitate more evenly throughout the tumor tissue, thus increasing the size of the Hot-Spots that are later generated and reducing the problem of tumor heterogeneity. On the other hand, if the diffusion away from the first enzyme was extreme, the diffusion could allow the soluble indoxyl molecules to diffuse into the blood or lymphatic capillaries where they could dimerize, precipitate, and deliver precipitates to normal tissues and reduce the subsequent radiation dose to the tumor. In order to obtain the advantage of controlled diffusion, and to circumvent the problem of the iadoxyl diffusing into the blood, various modifications can be made to the indoxyl-lactam so that the rate of diffusion of indoxyl into blood capillaries is greatly reduced Since charged molecules move much slower through the extra-cellular fluid than neutral molecules (Clauss and Jain, 1990, Cancer Research, 50, 3487-3493) (positively charged molecules tend to interact with negatively charged extra-cellular structures, and negatively charged materials are effectively repelled by the many negatively charged extra-cellular structures), molecules having a charge can be covalendy attached to the indoxyl-lactam to reduce the rate of diffusion of the soluble indoxyls and intermediates. This can be achieved by attaching a charged molecule to the benzene ring of the indoxyl by reductive amination, involving an amino group on the benzene ring and the reducing end (aldehyde) of the charged molecule. The result is an alkyl amino group, similar to that formed when polylysine is lactosylated by reductive amination. The resultant bond is incapable of being cleaved by mammalian enzymes, and the charged molecule will control the rate of movement of the released indoxyl to be optimum. The attachment of the charged molecule to the benzene ring of the indoxyl-lactam will not interfere with the ability of the beta-lactamase enzyme to cleave the indoxyl-lactam bond, or the ability of the indoxyl to be oxidized and to dimerize and precipitate.

A further modification can be made by covalently attaching two indoxyl-lactam molecules together at a position on the benzene ring to make a bi-indoxyl -lactam. Cleavage of the two lactam bonds of the bi-indoxyl-lactams by beta lactamase creates a bi-indoxyl molecule which will dimerize with two other bi-indoxyl molecules, and so on, to create a self-assembling linear insoluble polymer. The attachment of the two indoxyl -lactams can be either direct, or indirect via a digestible or non-digestible spacer molecule. The spacer molecule can be one of several kinds such as a poly (ethylene oxide) polymer with hetero-bifunctional reactive groups at its terminals (Yokoyama et al, 1992, Bioconjug. Chem. 3, 275-276), a non-degradable copolymer [N-(2-hydroxypropyl)methacrylamide] which is non-immunogenic, non-toxic, and has a versatile chemistry which allows for a range of side chains and pendant chemicals such as lactose, mannose, and radio-labeled tyrosinamide to be introduced (Maeda et al, 1992, Bioconug, Chem. 3,351-362; Seymour, 1992, Critical Reviews in Theautic Drug Carrier Systems, 9, 135-187; Primm et al, 1993, J. Drug Target 1, 125-131), or a hydrophobic hexamethylene spacer group (Ouchi et a1, 1992, Drug Des. Discov. 9, 93-105). The formation of an insoluble linear polymer has substantial advantages in reducing the ability of the substance to move by diffusion and convection in the extra-cellular fluid of the tumor tissue. Additional indoxyls can be made (a) by attaching chemicals to position 3 and which will precipitate by the action of the non-mammalian enzyme moiety of the bispecific reagent, (b) all substituents at position 4, 5, 6, and 7, including hydroxyl groups; (c) phenyl at position 5, and all its derivatives, (d) benzloxy at position 5 and all its derivatives and (e) 5.5-bi-indoxyls, with or without spacers.

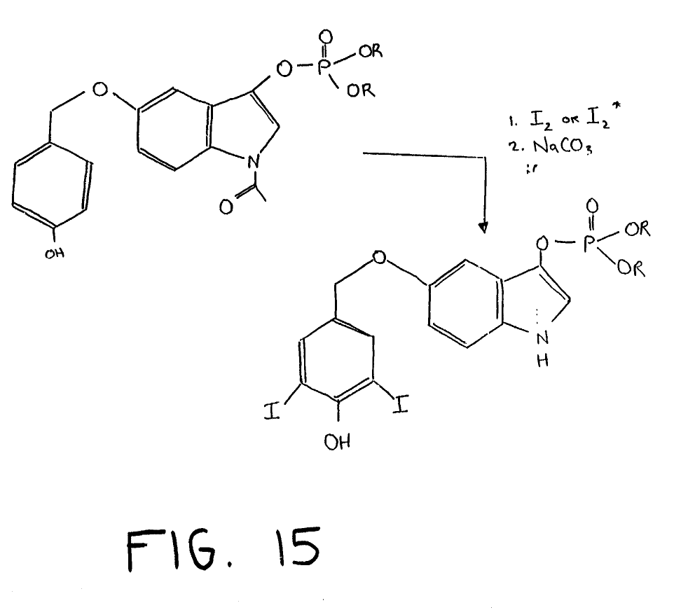

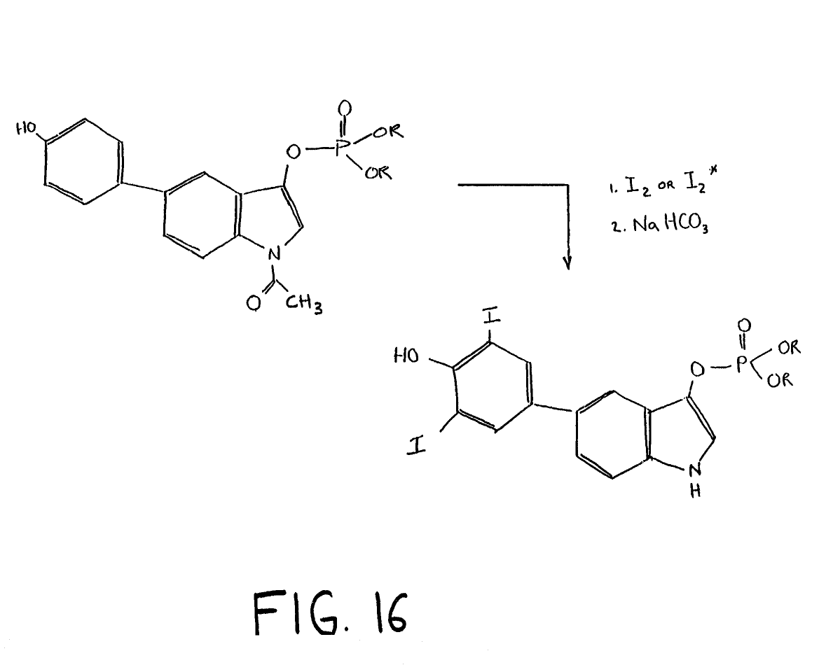

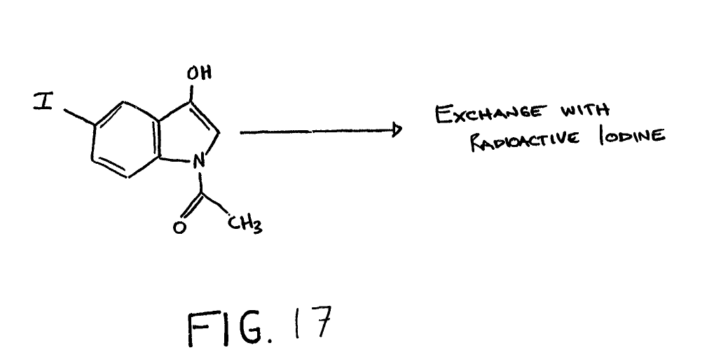

The first therapeutic agent can be radio-labeled to be trace-labeled or to be radio-active therapeutic. For example, the indoxyl can be radio-labeled with radioiodine or other radio-isotopes. Radio-iodination of the indoxyl compounds can be achieved in three ways: (i) FIG. 15 shows para-hydroxy Benzyl Ether of 5-Hydroxy-indoxyl phosphate is radio-iodinated, (ii) FIG. 16 shows indoxyl with a para-hydroxyphenyl substituted at the 5 position is radio-iodinated, and (iii) FIG. 17 shows the starting material, 1-acetyl-5-iodo-3-hydroxyindole, which is treated with radioactive iodine in 3M HCL. After warming for about 30 minutes, the iodoindole is re-isolated, now containing radioactive iodine. To prepare the tritium labeled compound, the corresponding indole is treated wits tritiated water under acid conditions in teterahydrofuran. The indole is re-isolated, in which the aromatic hydrogens have been replaced in part by tritium.

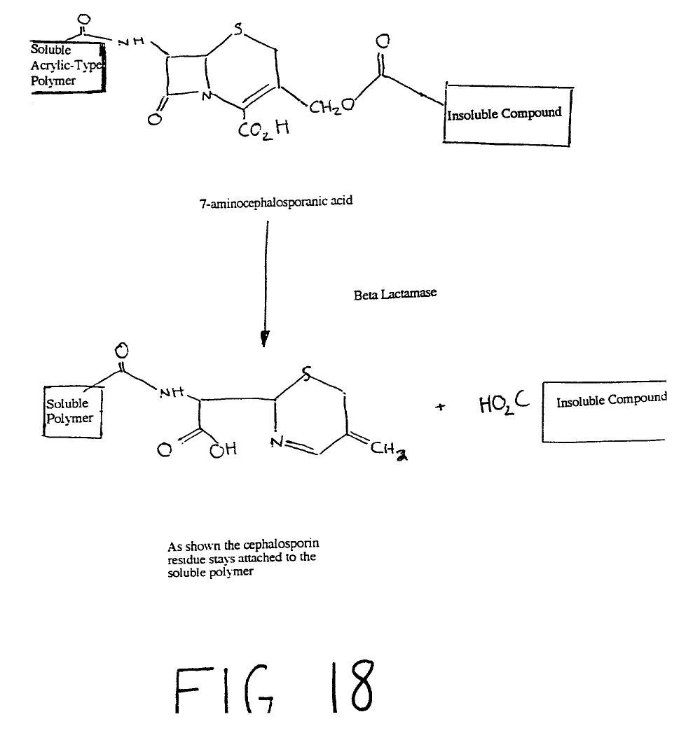

An additional method of converting first therapeutic agent into an insoluble material which precipitaes in the extra-cellular fluid is where the first therapeutic reagent has a soluble moiety and an insoluble moiety, the soluble moiety having a solubilizing effect on the insoluble moiety and being cleaved by the first enzyme moiety of the first bispecific reagent, the solubilizing effect of the soluble moiety being thereby dissipated and the remaining material, being insoluble, spontaneously forming a precipitate. FIG. 18 shows an specific example of this method of precipitation in which beta lactamse cleaves the bond between the soluble and insoluble moiety causing the insoluble moiety to spontaneously precipitate.

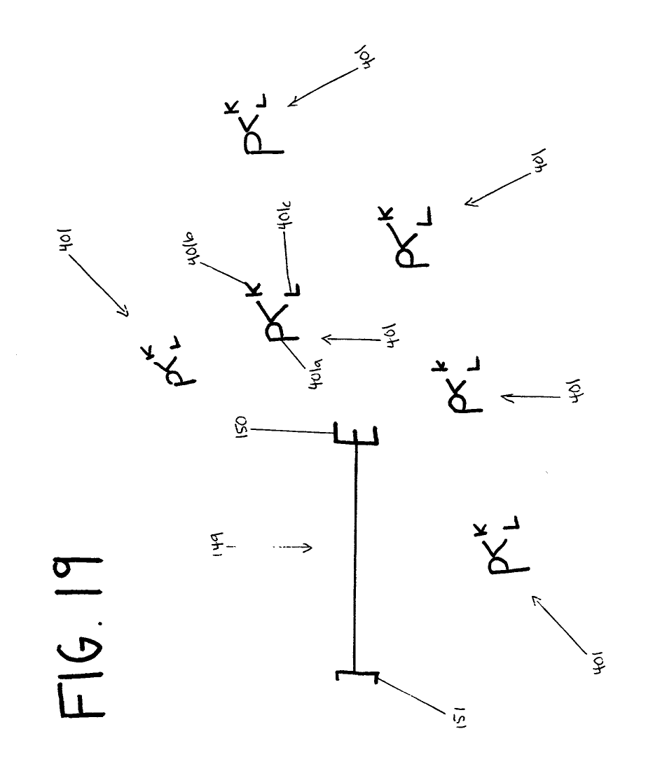

In accordance with the method of the present invention, continuing the introducing of the first therapeutic agent into the living host increases the amount of the accumulation of the first extra-cellular precipitate in the extra-cellular fluid to form a plurality of antigenic epitopes adjacent to the first bispecific reagent which is proportional to the amount of accumulation. As shown in FIG. 19, the continued introducing of the first therapeutic agent increases the amount of precipitate 401 which accumulates adjacent to the first bispecific reagent 149 in the extra-cellular fluid. The first extra-cellular precipitate having at least one of a first antigenic epitope 401a, a second antigenic epitope 401b, and a neo-antigenic third epitope 401c, the accumulation of first extra-cellular precipitate thereby forming a plurality of antigenic epitopes.

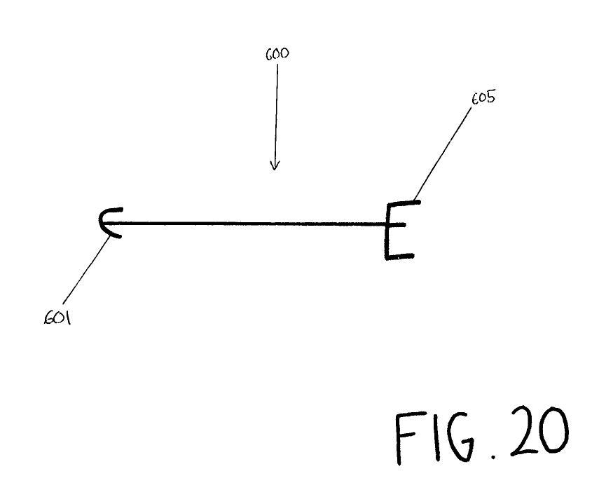

The method of the present invention includes additionally introducing into the living host a second bispecific reagent FIG. 20 shows the second bispecific reagent 600 which is introduced to the living host The second bispecific reagent, is adapted to be retained at the first extra-cellular precipitate. The second bispecific reagent having two moieties, the first moiety being a non-mammalian enzyme moiety being the second enzyme moiety 605 The second moiety of the second bispecific reagent including a targeting agent moiety 601, which is adapted to have a substantial affinity for at least one of the first antigenic epitope, the second antigenic epitope, and the neo-antigenic third epitope of the first extra-cellular precipitate. The second enzyme moiety being adapted to convert a substantial amount an additional therapeutic agent into a new form capable of remaining in the extra-cellular fluid adjacent to the first extra-cellular precipitate for an extended period of time sufficient to kill non-selectively all cells adjacent to the first extra-cellular precipitate.

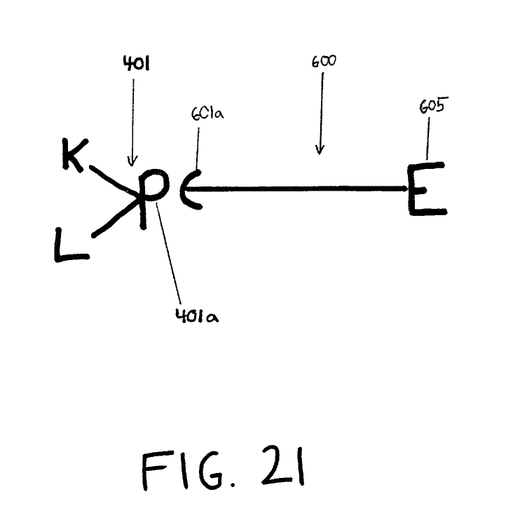

FIG. 21 shows an example of the second bispecific reagent 600 which has two moieties. The first moiety of the second bispecific reagent being the second enzyme moiety 605, the second moiety of the second bispecific reagent being a targeting agent moiety 601a having an affinity for the first antigenic epitope of the extra-cellular precipitate, bound to the first antigenic epitope 40la of the extra-cellular precipitate 401.

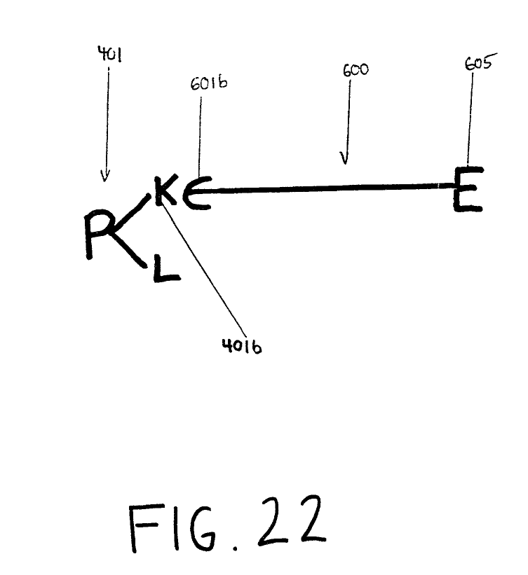

FIG. 22 shows an example of the second bispecific reagent 600 having two moieties, the first moiety of the second bispecific reagent being the second enzyme moiety 605, the second moiety of the second bispecific reagent being a targeting agent moiety 601b having an affinity for the second antigenic epitope of the extra-cellular precipitate, bound to the second antigenic epitope 401b of the extra-cellular precipitate 400.

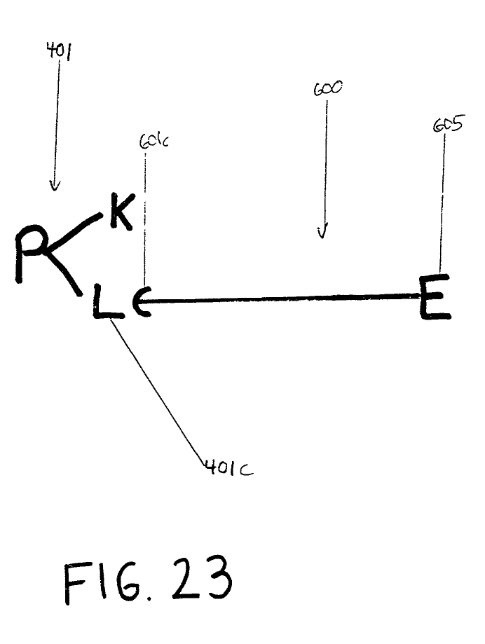

FIG. 23 shows an example of the second bispecific reagent 600 having two moieties, the first moiety of the second bispecific reagent being the second enzyme moiety 605, the second moiety being a targeting agent moiety 601c having an affinity for the neo-antigenic third epitope of the extra-cellular precipitate, bound to the neo-antigenic third epitope 401c of the extra-cellular precipitate 400. As FIG. 21, 22, and 23 show, following the delivery of the second bispecific reagent 600, the second bispecific reagent is received and bound at the plurality of the antigenic epitopes of the first extra-cellular precipitate 400.

Following the delivery to the living host of the second bispecific reagent, some of the second bispecific reagent will be in body fluids or bound non-specifically to non-target cells or to extra-cellular structures. It is known that, with time, circulating antibodies and antibodies bound non-specifically, are naturally eliminated from the body of the living host more quickly than antibodies that are bound to specific targets (Henkel et al., 1985, Clinical Immunology and Immunopathology, 35, 146-155: Goldberg et al., 1988, Archives Of Pathology Laboratory Medicine, 112, 580-587). Therefore, the elimination of the second bispecific reagent from the living host can be achieved simply by allowing a lapse of time; the elimination of the second bispecific reagent can be hastened, as has been described previously for the second step of the ADEPT process (Sharma et al, 1994, Cancer Supplement, 73, 1114-1120), by the administration of a galactosylated antibody specific for the second enzyme moiety of the second bispecific reagent. The galactosylated antibody can inactivate the enzyme and/or facilitate the clearance of the second enzyme moiety-galactosylated antibody complexes via the galactose specific receptors in the liver.

The elimination, from the living host, of all the second bispecific reagent not specifically bound to the first extra-cellular precipitate prior to the administration of the additional therapeutic agent is necessary to prevent the additional therapeutic agent from being converted into the new form in locations other than in the tumor. After all unbound second bispecific reagent has been eliminated from the living host, the additional therapeutic agent is additionally administered to the living host, where it circulates freely in the body fluids and is converted by the second enzyme moiety of the second bispecific reagent into a new form.

In accordance with the method of the present invention, after all unbound second bispecific reagent has been eliminated from the living host, the additional therapeutic agent, which is a soluble radioactive toxic agent, is additionally administered to the living host, the additional therapeutic agent adapted to be converted, in the extra-cellular fluid adjacent to the first extra-cellular precipitate, by the second enzyme moiety of the second bispecific reagent bound to the first extra-cellular precipitate, into a radioactive toxic new form. The new form of the additional therapeutic agent being capable of remaining in the extra-cellular fluid adjacent to the first extra-cellular precipitate for an extended period of time sufficient to kill non-selectively all cells adjacent to the first extra-cellular precipitate. Since the first extra-cellular precipitate is retained in the extra-cellular fluid for an extended period of time and since the second bispecific reagent is bound thereto, the continued administration of the additional therapeutic agent enables the second enzyme moiety to convert a substantial amount of the additional therapeutic agent into the radioactive toxic new form. The radioactive toxic new form remaining in the extra-cellular fluid adjacent to the first extra-cellular precipitate for an extended period of time thereby creating an intense field of radiation—called a Hot-Spot—which kills non-selectively all cells adjacent to the fist extracellular precipitate.

Since the enzymatic conversion of the additional therapeutic agent takes place exclusively in the extra-cellular fluid, there is no need for the additional therapeutic agent to gain entrance into the intra-cellular compartment of the body. Entrance of the additional therapeutic agent into cells throughout the living host would increase the volume of distribution of the additional therapeutic agent (the intra-cellular compartment has a volume at least 2-3 times that of the extra-cellular fluid) and would increase the duration of time that the additional therapeutic agent circulates in the living host. Both these factors—the increased volume of distribution and the increased circulation time—would increase the systemic toxicity caused by the additional therapeutic agent. For these reasons it is of significant advantage for the additional therapeutic agent to be cell impermeant by being a molecule larger than 1000 daltons and/or being anionic. Alternatively the additional therapeutic agent can be made cell impermeant by attaching one of a number of cell impermeant molecules at least including peptides or polymers having a molecular size greater than 1,000 daltons and anionic chemicals including thiols.

In accordance with the present invention, there are at least three different methods of retaining the new form of the additional therapeutic agent in the tumor region. Each method of the invention involves the step of delivering into the living host the second bispecific reagent and additionally administering to the living host the additional therapeutic agent which is converted by the second enzyme moiety of the second bispecific reagent into a new form which is retained for an extended period of time in the extra-cellular fluid adjacent to the first extra-cellular precipitate by at least three different methods.

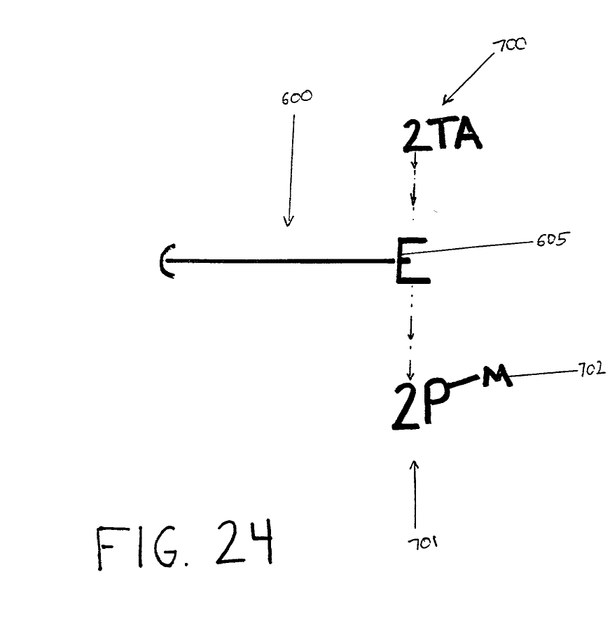

As shown in FIG. 24, in the first method of retaining the new form of the additional therapeutic agent in the extra-cellular fluid of the tumor region, the soluble radioactive toxic additional therapeutic agent, being the second therapeutic agent 700 is converted by the second enzyme moiety 605 of the second bispecific reagent 600 into a new form 701 which is insoluble and which spontaneously forms a radioactive toxic precipitate being the second extra-cellular precipitate 701 and having a neo-antigenic epitope 702 not found on the second therapeutic agent 700, the neo-antigenic epitope 702 enabling the second extra-cellular precipitate 701 to be “tethered” via a previously administered bispecific reagent bound to stable structures in the tumor tissue (as described later). The tethering of the second extra-cellular precipitate thereby causing the second extra-cellular precipitate to be retained for an extended period of lime in the extra-cellular fluid adjacent to the first extra-cellular precipitate thereby enabling the radioactive toxic second extra-cellular precipitate to generate an intense field of radiation, called a Hot-Spot, which kills non-selectively all cells adjacent to the first extra-cellular precipitate.

The second therapeutic agent to be used in the first method can be a soluble radioactive toxic precipitable agent made by converting chemical X to a soluble XY. The bond attaching X to Y is cleaved by the second enzyme moiety of the second bispecific reagent to create the highly reactive intermediate molecule Xa. The Xa molecule is readily and extremely rapidly oxidized to form Xb. In the oxidized form, Xb spontaneously and covalently self-condenses or dimerizes to create a new molecule which is insoluble and immediately and spontaneously forms a radioactive toxic second precipitate. Because a new molecule is formed by the dimerization, the core structure of the second precipitate has a neo-antigenic epitope which is not present on the original XY, Xa, Xb or anywhere else in the body.

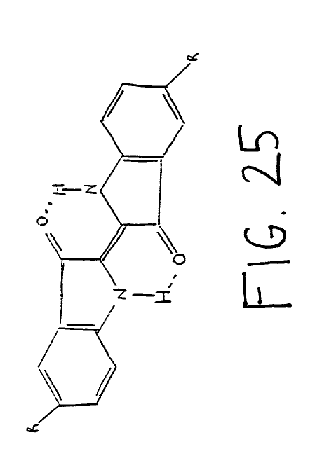

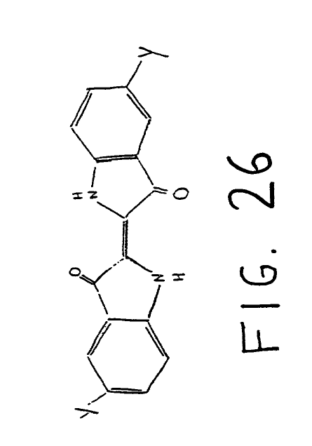

A specific example of a second therapeutic agent to be used in the first method is the application of a radioactive indoxyl-lactam as examples of XY, which is soluble and can be administered to the living host as a free molecule. The lactam (Y) of the indoxyl-lactam (XY) is cleaved by beta-lactamase enzyme (being the enzyme moiety of the bound bispecific reagent) to liberate a highly reactive intermediate indoxyl (Xa). As shown in FIG. 25 the indoxyl (Xa) is readily and extremely rapidly oxidized, and once in he oxidized form it spontaneously self-condenses or dimerizes to form a new molecule which is insoluble and precipitates spontaneously as a radioactive toxic second extra-cellular precipitate being a radioactive toxic indigo dye as illustrated in FIG. 26 where Y can be aryl, halogen, hydoxyl, and alkyl. The new molecule is different from the indoxyl-lactam and the intermediate indoxyl molecules and thereby having a neo-antigenic epitope not present on the indoxyl-lactam or the indoxyl intermediate.

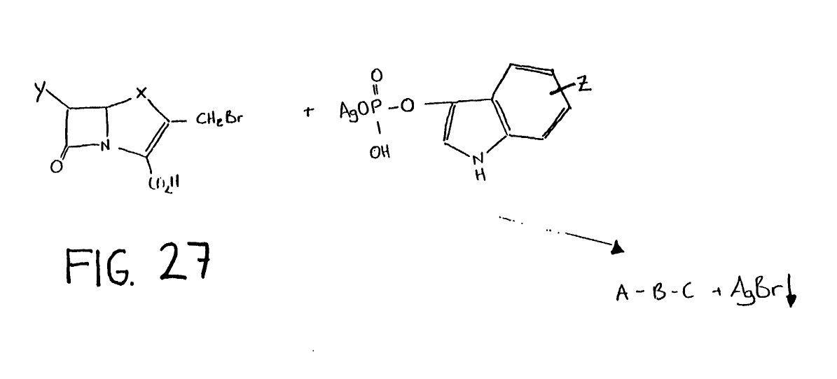

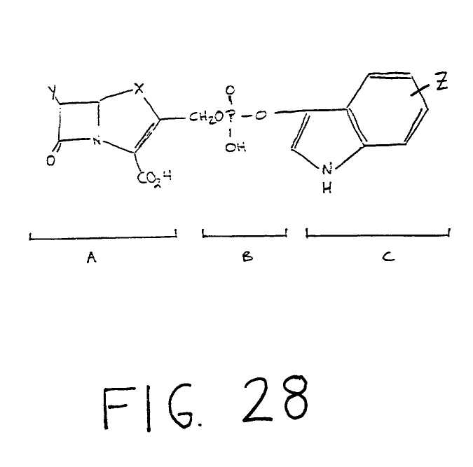

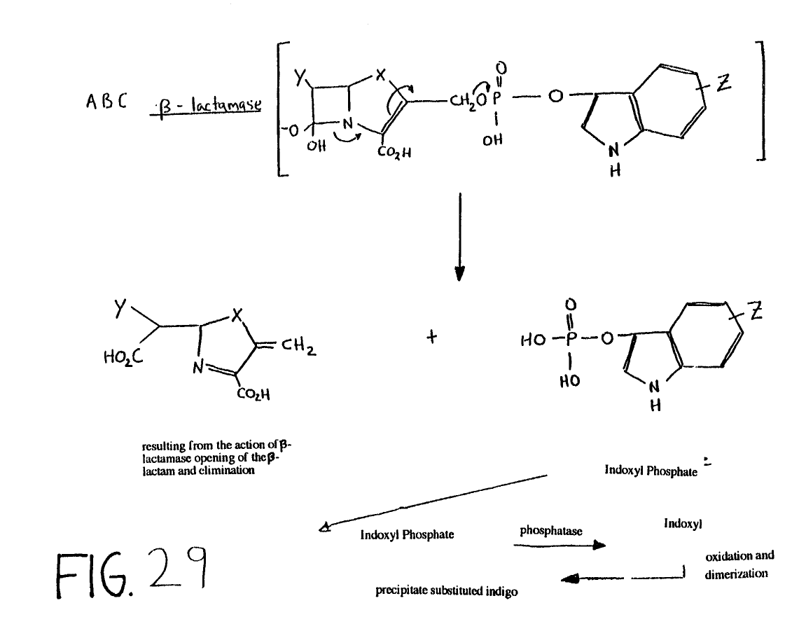

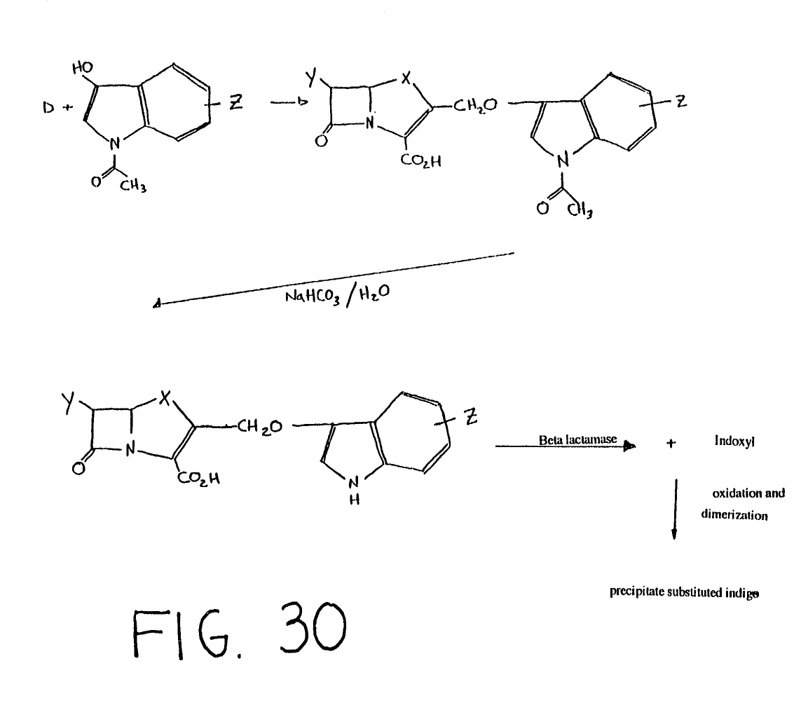

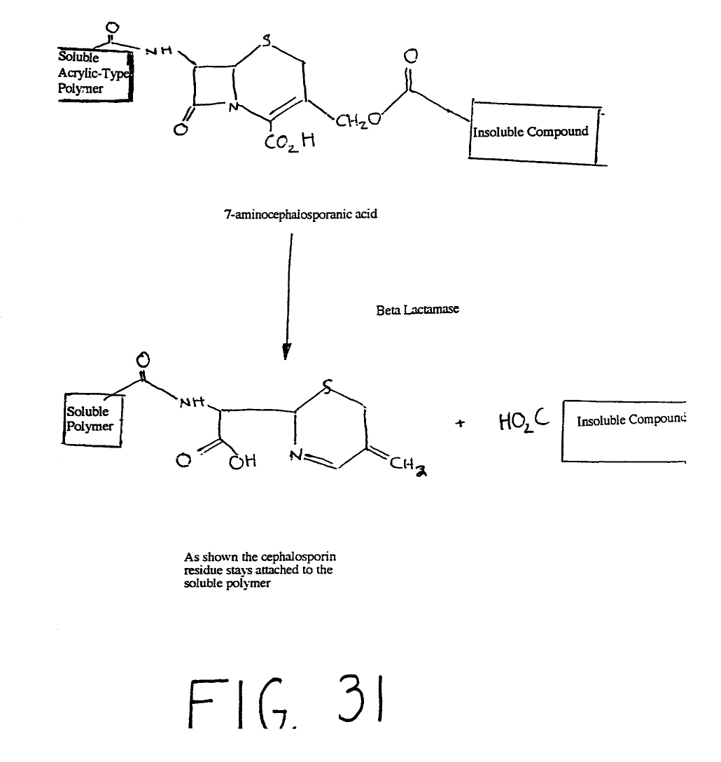

FIG. 27 and FIG. 28 where Y is a typical penicillin at the 6 position, where X is oxygen, sulfur, or carbon, where Z is an appropriate substitute in the indoxyl which allows attachment to targeting agent, alternatively attachment to the targeting agent may be effected through Y, where B is the phosphate (as will be shown similar events may occur with or without the phosphate group), and where C is the substituted indoxyl portion that, when liberated, dimerizes and precipitates. As shown in these diagrams, the lactam can be attached to position 3 of the indoxylphosphate via the phosphate group, in which case it is detached by beta-lactamase to form indoxylphosphate. As shown in FIG. 29 and in FIG. 30 the phosphate group of the remaining indoxylphosphate is cleaved by phosphatase naturally present in body fluids to produce indoxyl which passes through the steps previously described to precipitate. FIG. 31 illustrates how the lactam can also be attached directly to the indoxyl at position 3, in which case precipitation occurs by the direct action of beta-lactamase.

The oxidation and dimerization of indoxyl proceeds at a slower rate in the acidic pH which is often present in the extra-cellular fluid of the tumor tissue, compared to the rate of oxidation and dimerization in the relatively neutral pH found in the extra-cellular fluid of normal tissues. The slower rate of oxidation and dimerization may allow some of the soluble indoxyl molecules and intermediates to diffuse away from the second enzyme moiety of the second bispecific reagent prior to the indoxyl oxidizing, dimerizing and precipitating. A controlled diffusion away from the second enzyme moiety would have the advantage of distributing the radioactive toxic second precipitate more evenly throughout the tumor tissue, thus increasing the size of the Hot-Spots and reducing the problem of tumor heterogeneity. On the other hand, if the diffusion away from the second enzyme moiety was extreme, it could allow the soluble indoxyl molecules to diffuse into the blood or lymphatic capillaries where it could dimerize, precipitate, and deliver radioactive precipitates to normal tissue and reduce the radiation dose to the tumor. In order to obtain the advantage of controlled diffusion, and to circumvent the problem of the indoxyl diffusing into the blood, various modifications can be made to the indoxyl -lactam so that the rate of diffusion of indoxyl into blood capillaries is greatly reduced. Since charged molecules move much slower through the extra-cellular fluid than neutral molecules (Clauss and Jain, 1990, Cancer Research, 50, 3487-3493) (positively charged molecules tend to interact with negatively charged extra-cellular structures, and negatively charged materials are effectively repelled by the many negatively charged extra-cellular structures), molecules having a charge can be covalently attached to the indoxyl-lactam to reduce the rate of diffusion of the soluble indoxyls and intermediates. This can be achieved by attaching a charged molecule to the benzene ring of the indoxyl by reductive amination, involving an amino group on the benzene ring and the reducing end (aldehyde) of the charged molecule. The result is an alkyl amino group, similar to that formed when polylysine is lactosylated by reductive amination. The resultant bond is incapable of being cleaved by mammalian enzymes, and the charged molecule will control the rate of movement of the released indoxyl to be optimum. The attachment of the charged molecule to the benzene ring of the indoxyl-lactam will not interfere with the ability of the beta-lactamase enzyme to cleave the indoxyl-lactam bond, or the ability of the indoxyl to be oxidized and to dimerize and precipitate.

A further modification can be made by covalently attaching two indoxyl-lactam molecules together at a position on the benzene ring to make a bi-indoxyl-lactam. Cleavage of the two lactam bonds of the bi-indoxyl-lactams by beta lactamase creates a bi-indoxyl molecule which will dimerize with two other bi-indoxyl molecules, and so on, to create a self-assembling linear insoluble polymer. The attachment of the two indoxyl-lactams can be either direct, or indirect via a digestible or non-digestible spacer molecule. The spacer molecule can be one of several kinds such as a poly (ethylene oxide) polymer with hetero-bifunctional reactive groups at its terminals (Yokoyama et al, 1992, Bioconjug. Chem. 3, 275-276), a non-degradable copolymer [N-(2-hydroxypropyl)methacrylamide] which is non-immunogenic, non-toxic, and has a versatile chemistry which allows for a range of side chains and pendant chemicals such as lactose, mannose, and radio-labeled tyrosinamide to be introduced (Maeda et al, 1992, Bioconug, Chem. 3, 351-362; Seymour, 1992, Critical Reviews in Therapeutic Drug Carrier Systems, 9, 135-187; Primm et al, 1993, J. Drug Target 1, 125-131), or a hydrophobic hexamethylene spacer group (Ouchi et al, 1992, Drug Des. Discov. 9, 93-105). The formation of an insoluble linear polymer has substantial advantages in reducing the ability of the substance to move by diffusion and convection in the extra-cellular fluid of the tumor tissue. Additional indoxyls can be made (a) by attaching chemicals to position 3 and which will precipitate by the action of the non-mammalian enzyme moiety of the bispecific reagent, (b) all substituents at position 4, 5, 6, and 7, including hydroxyl groups; (c) phenyl at position 5, and all its derivatives, (d) benzloxy at position 5 and all its derivatives and (e) 5,5-bi-indoxyls, with or without spacers.

An additional method of converting a soluble second therapeutic agent into an insoluble material which precipitaes in the extra-cellular fluid is where the second therapeutic reagent has a soluble moiety and an insoluble moiety, the soluble moiety having a solubilizing effect on the insoluble moiety. The soluble moiety being cleaved by the non-mammalian enzyme of the bound bispecific reagent, the solubilizing effect of the soluble moiety being thereby dissipated and the remaining material, being insoluble, spontaneously forming a precipitate. FIG. 31 shows an specific example of this method of precipitation in which beta lactamse cleaves the bond between the soluble moiety and insoluble moiety causing the insoluble moity to spontaneously precipitate.

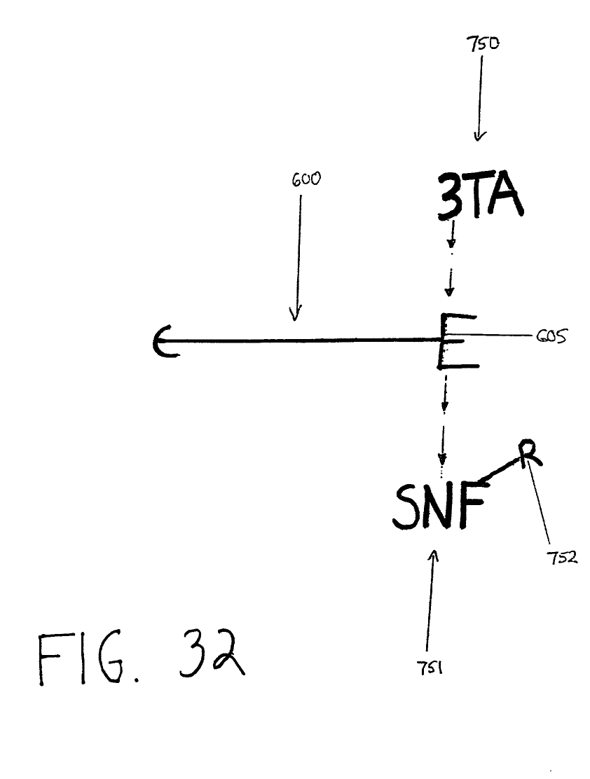

As shown in FIG. 32, according to the second method of retaining the new form of the additional therapeutic agent in the tumor region, the additional therapeutic agent being the third therapeutic agent 750 is converted by the second enzyme moiety 605 of the second bispecific reagent 600 into a new form 751 which is soluble and has a neo-antigenic epitope 752, the neo-antigenic epitope 752 not being present on the third therapeutic agent 750. The neo-antigenic epitope 752 of the soluble new form of the third therapeutic agent 751 being used, as described later, to “tether” the new form of the third therapeutic agent 751 via previously administered bispecific reagents which are bound to stable structures in the tumor tissue. The tethering retains the new form of the radioactive toxic third therapeutic agent adjacent to the first extra-cellular precipitate for an extended period of time, the new form of the third therapeutic agent thereby generating intense fields of radiation, called Hot-Spots, which kill non-selectively all cells adjacent to the first extra-cellular precipitate.

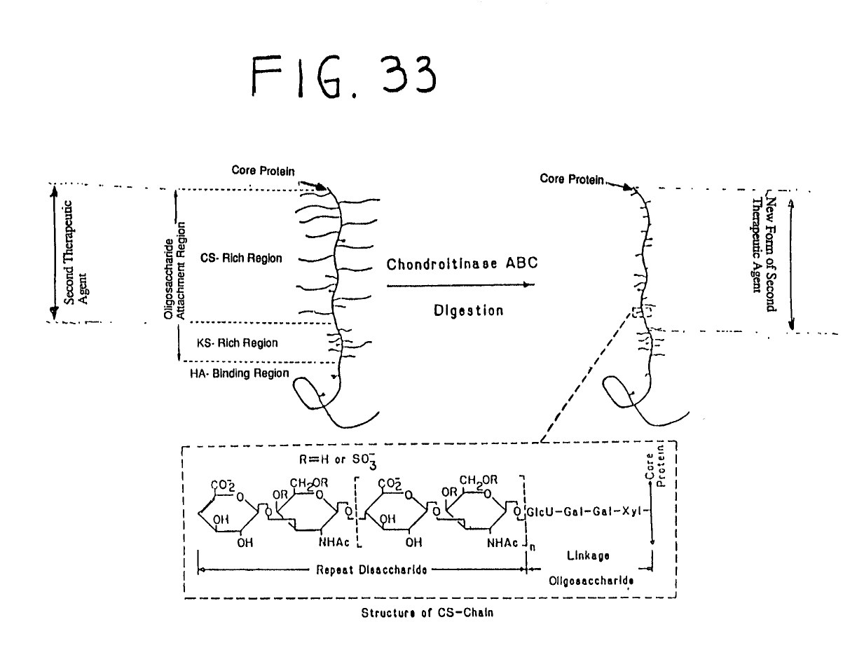

A specific example of a non-mammalian enzyme-substrate system to be used for this method is chondroitinase ABC as the second enzyme moiety, and radio-labeled chondroitin sulphate attached to a short polypeptide as the third therapeutic agent, as shown in FIG. 33. Chondroitin sulphate is degraded by the chondroitinase ABC enzyme which cleaves the repeat disaaharide portion of the chondroitin sulphate chain and to leave only the linkage oligosaccharide with its terminal glucuronic acid residue attached to the protein core. The chondroitin sulphate is thereby converted by the chondroitinase ABC enzyme into a new form which is soluble and has a neo-antigenic epitope not found on the untreated chondroitin sulphate (Haskall et al, 1972, J. Biol. Chem., 247, 4521-4528; Distler and Jourdian 1973, J. Biol. Chem., 248, 6772-6780). The new form of the third therapeutic agent is soluble and is tethered via its neo-antigenic epitope by previously administered bispecific reagents bound to stable structures in the tumor tissue for an extended period of time as described later. The retaining of the new form of the third therapeutic agent in the extra-cellular fluid adjacent to the first extra-cellular precipitate thereby generating intense fields of radiation, called Hot-Spots, which kill non-selectively all cells adjacent to the first extra-cellular precipitate.

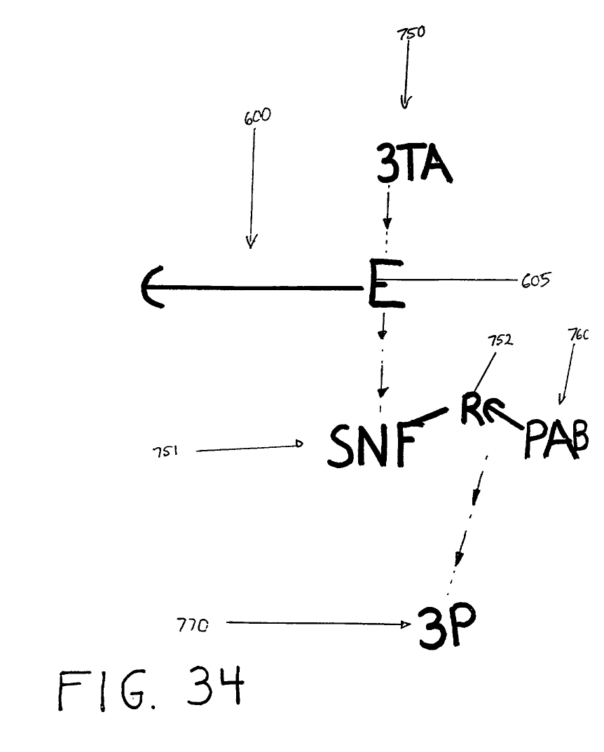

As illustrated in FIG. 34, according to the third method of retaining the new form of the additional therapeutic agent in the tumor region, the soluble radioactive toxic additional therapeutic agent, being the third therapeutic agent 750 is converted by the second enzyme moiety 605 of the second bispecific reagent 600 into a new form 751 which is soluble and has a neo-antigenic epitope 752 not present on the third therapeutic agent 750. Prior to administering the third therapeutic agent 750, a precipitating antibody 760 which is adapted to react with the neo-antigenic epitope 752 of the new form of the third therapeutic agent 751 is administered to the living host. The precipitating antibody 760 has the ability to bind to the neo-antigenic epitope 752 of the new form of the third therpeutic agent 751, the binding causing a precipitate to form, the precipitate being the third extra-cellular precipitate 770 which is composed of the administered precipitating antibody 760 complexed to the new form of the third therapeutic agent 751.

The administration of large molecules results in higher concentrations of large molecules to be present in tumor tissue compared to normal tissue (Seymour, 1992, Critical Reviews in Therapeutic Drug Carrier Systems, 91, 135-187). Therefore, the concentration of the administered precipitating antibody, being a large molecule, will be higher in the tumor than in normal tissues. The higher concentration of the precipitating antibody in tumor tissues enables a larger amount of the new form of the third therapeutic agent to bind and complex to the precipitating antibody and thereby form a precipitate, being the third extra-cellular precipitate which is retained in the extra-cellular fluid adjacent to the first extra-cellular precipitate for an extended period of time.

A specific example of a non-mammalian enzyme-substrate system to be used in the third method is chondroitinase ABC as the second enzyme moiety, and radio-labeled chondroitin sulphate (CS) as the additional therapeutic agent. As described in method two, the chondroitinase ABC converts the chondroitin sulphate into a new form, the new form being a soluble material and having a neo-antigenic epitope not found on the chondroitin sulphate (Christner et al, 1980, J. Biol. Chem., 255, 7102-7105). Prior to the administration of the chondroitin sulphate, a precipitating antibody capable of binding to the neo-antigenic epitope of the chondroitin sulphate is administered to the living host. The complex, formed by the administered precipitating antibody binding to the neo-antigenic epitope of the chondroitin sulphate forms a precipitate, the precipitate being the third extra-cellular precipitate which remains in the extra-cellular fluid adjacent to the first extra-cellular precipitate for an extended period of time sufficient to kill non-selectively all cells adjacent to the first extra-cellular precipitate.

The intensity of the radiation field that will be generated by each of the three methods that use the second enzyme moiety to convert the additional therapeutic agent into a new form capable of generating Hot-Spots is very high. A large number second enzyme moiety molecules, for example beta-lactamase or chondroitinase ABC, will be bound to the first extra-cellular precipitate via the first bispecific reagent. The number of enzyme molecules is proportional to the amount of precipitate which had accumulated in the extra-cellular fluid adjacent to the first extra-cellular precipitate. Since it is possible to accumulate any required amount of first extra-cellular precipitate, the number of bound second bispecific reagents can be made very large, therefore, the number of second enzyme moiety molecules can also be made very large. The more second enzyme moiety molecules that are present, the higher will be the rate at which the additional therapeutic agent is converted into the new form. The higher the rate of conversion, the more intense the radiation field adjacent to the first extra-cellular precipitate will be and less intense the level of systemic toxicity in the living host will be.

The present invention exploits a number of naturally occurring biological mechanisms of the loving host to increase the specificity of the location of Hot-spots to the tumor. Following the administration of the first therapeutic agent, and prior to the delivery of the bispecific reagent and/or the additional administration of the second therapeutic agent, several mechanisms operate to transfer first extra-cellular precipitate in normal tissue (but not cancer tissue) to a location where first extra-cellular precipitate cannot act as a platform from which a Hot-Spot can later be generated. Macrophages in the normal tissue can transfer the first extra-cellular precipitate to an intra-cellular location by phagocytosis, thereby preventing Hot-Spots from developing. In contrast, macrophages in cancer tissue are inhibited by the cancer cell driven aberrant environment (Boetcher and Leonard, 1974, J. Nat. Cancer Inst. 52, 1091-1096; Snyderman et al, 1978, J. Nat. Cancer Inst. 60, 737-742; Norman, 1985, in Macrophage Biology, p.285-298, Alan R Liss Inc.; Braun et al, 1993, Cancer Research, 53, 3362-3368) and will not phagocytose the first extra-cellular precipitate as effectively.

In addition, normal epithelial cells that line the boundary between the inner and outer environment of the body exfoliate into the lumen of the organ when they are killed (Ishikaawa et al, 1993, 17 suppl. pS 104-110; Montefort et al, 1993, Eur. Respir. J. , 6, 1257-1263; Sisson et al, 1994, Am. J Respir. Crit. Care Med. 149, 205-213). The exfoliation of these cells effectively transfers the first bispecific reagent and any first extra-cellular precipitate to a location where the first extra-cellular precipitate cannot act as a platform from which Hot-Spots can later be generated. In contrast, cancer cells which arise from these boundary cells only grow inside the body of the host and cannot exfoliate to the external environment. Consequently, virtually all cancer epithelial cells (but not normal epithelial cells) that have received the first bispecific reagent and which have generated a first extra-cellular precipitate will be in a correct location to generate Hot-Spots.

Similarly, normal endothelial cells that are damaged or killed become detached and enter the blood stream (Dini et al, 1995, J. Cell. Sci. 108, 967-73) and any bispecific reagent bound to them will be engulfed quickly by the macrophages which line the sinusoids of the liver and spleen, thereby making the first extra-cellular precipitate unavailable to generate Hot-Spots from.

Particles which are injected into the extra-cellular fluid move by convective flow from the extra-cellular fluid of tissues into the lymph drainage channels which drain into to the regional lymph nodes, where the particles are quickly and effectively engulfed by the very active macrophages which line the lymph flow pathway. First extra-cellular precipitates and second and third extra-cellular precipitates behave in a similar way and suffer the same fate. The movement of precipitates can occur in normal tissue thereby reducing the number of Hot-Spots which would otherwise have been generated in normal tissue. The movement of the first extra-cellular precipitate in normal tissue enables the first extra-cellular precipitate to be engulfed by macrophages in the regional lymph glands prevents Hot-Spots from developing in normal tissues, whereas movement of radioactive second and third precipitates in normal tissues which enables the radioactive precipitates to be engulfed by macrophages in the regional lymph glands causes Hot-Spots to be generated in the regional lymph glands, which is a much more desirable and clinically safe location for radiation damage to occur, compared to normal parenchymal tissue. In contrast, cancer tissue lacks an effective lymphatic drainage system (Jain, 1987, Cancer Research, 47, 3039-3051; Jain and Baxter, 1988, Cancer Research, 48, 7022-7032; Clauss and Jain, 1990, 50, 3487-3497) and first, second, and third extra-cellular precipitate movement into the lymphatic system cannot take place from tumor tissue and there will be no reduction in the number of Hot-Spots which will be generated in tumor tissue.

Lymphatic vessels are sometimes present in tumor tissue (reflecting the heterogeneity of non-malignant cells within the tumor tissue) which could lead to movements of the first, second and third extra-cellular precipitate into regional lymph glands. This movement can be prevented by “tethering” the first and second extra-cellular precipitate and the soluble new form of the second therapeutic agent to stable structures which are substantially more present in tumor tissue. Tethering of the first extra-cellular precipitate can be achieved by administering bispecific reagents to the living host prior to the administration of the additional therapeutic agent. The bispecific reagents tether the extra-cellular precipitate to at least one of three different stable structures in the extra-cellular fluid including the third antigenic receptor of the second target cancer cells, the antigenic epitopes of the cancer-altered extra-cellular matrix, and the antigenic epitopes of the relocated natural intra-cellular material. The tethering bispecific reagents are comprised of two moieties, the first moiety having an affinity for one of the first antigenic epitope, second antigenic epitope, and neo-antigenic third epitope of the extra-cellular precipitate. The second moiety having an affinity for at least one of three different stable structures in the extra-cellular fluid including the third antigenic receptor of the second target cancer cells, the antigenic epitopes of the cancer-altered extra-cellular matrix, and the antigenic epitopes of the relocated natural intra-cellular material.

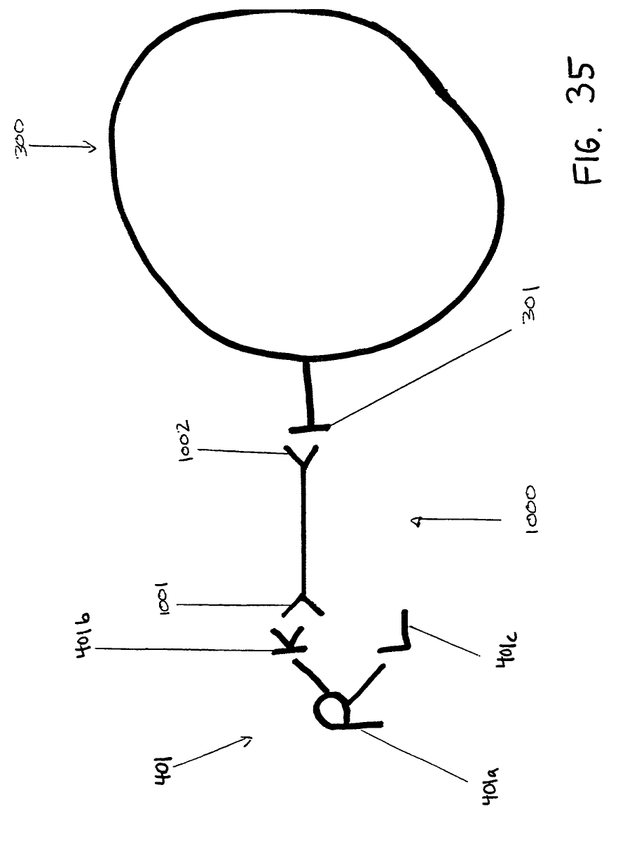

FIG. 35 shows the third bispecific reagent 1000 tethering the first extra-cellular precipitate 401 to the second antigenic receptor 301 of the second target cancer cells 300. The third bispecific reagent 1000 being comprised of two moieties, the first moiety 1001 being a targeting agent with an affinity for the second antigenic epitope 401b of the first extra-cellular precipitate 400. The second moiety 1002 of the third bispecific reagent being capable of binding to the third antigenic receptor 301 of the second target cancer cells 300. The third bispecific reagent thereby tethering the first extra-cellular precipitate 401 and retaining it in the extra-cellular fluid of the cancer. Alternatively the first moiety of the third bispecific reagent 1001 could have an affinity for the first antigenic epitope 401a of the first extra-cellular precipitate 400 or could have an affinity for the neo-antigenic third epitope 401c of the first extra-cellular precipitate 400.

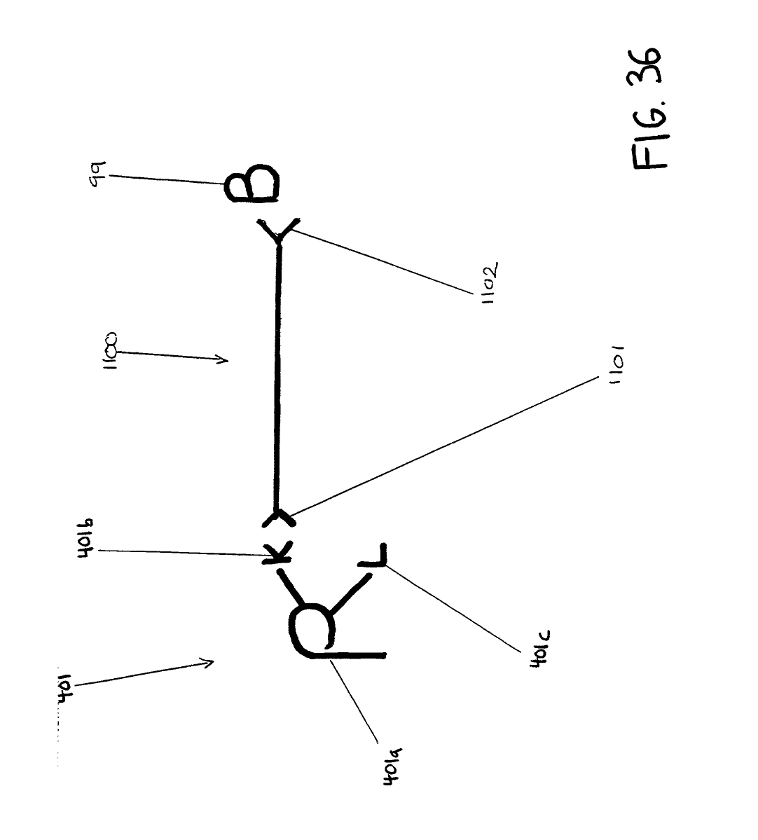

FIG. 36 shows the fourth bispecific reagent 1100 tethering the first extra-cellular precipitate 401 to the antigenic epitopes of the cancer-altered extra-cellular matrix 99. The fourth bispecific reagent 1100 being comprised of two moieties, the first moiety 1101 being a targeting agent with an affinity for the second antigenic epitope 401b of the first extra-cellular precipitate 155. The second moiety 1102 of the fourth bispecific reagent being capable of binding to the antigenic epitopes of the cancer-altered extra-cellular matrix 99. The fourth bispecific reagent thereby tethering the first extra-cellular precipitate 401 and retaining it in the extra-cellular fluid of the cancer. Alternatively the first moiety 1101 of the fourth bispecific reagent could have an affinity for the first antigenic epitope 401a of the first extra-cellular precipitate 401 or could have an affinity for the neo-antigenic third epitope 401c of the first extra-cellular precipitate 401.

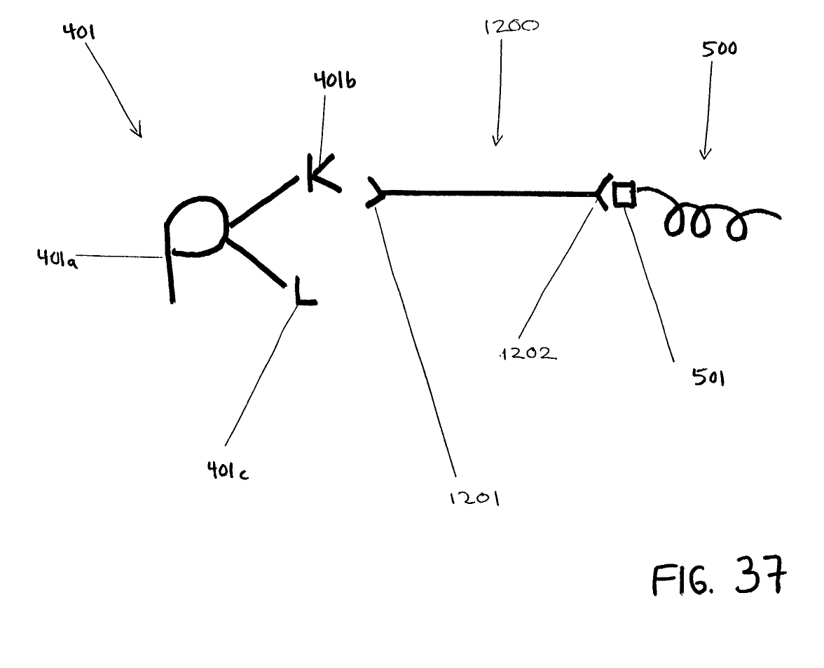

FIG. 37 shows the fifth bispecific reagent 1200 tethering the first extra-cellular precipitate 401 to the antigenic epitopes of the relocated natural intra-cellular material 501. The fifth bispecific reagent 1200 being comprised of two moieties, the first moiety 1201 being a targeting agent with an affinity for the second antigenic epitope 401b of the first extra-cellular precipitate 400. The second moiety 1202 of the fifth bispecific reagent being capable of binding to the antigenic epitopes of the relocated natural intra-cellular material 501. The fifth bispecific reagent thereby tethering the first extra-cellular precipitate 401 and retaining it in the extra-cellular fluid of the cancer. Alternatively the first moiety 1201 of the fifth bispecific reagent could have an affinity for the first antigenic epitope 401a of the first extra-cellular precipitate 401 or could have an affinity for the neo-antigenic third epitope 401c of the first extra-cellular precipitate 401.

Specificity of Hot-Spot location is also increased because the binding moiety of the second bispecific reagent is adapted to bind exclusively to the antigenic epitopes of the first extra-cellular precipitate and the first extra-cellular precipitate is a material not naturally present in the body, therefore, the binding moiety of the second bispecific reagent with its second enzyme moiety can have a high and specific affinity for the antigenic epitopes of the first extra-cellular precipitate with little or no cross reaction to natural structures of the living host. For example, the cellulose binding domain peptide, from which the second bispecific reagent can be made binds virtually irreversibly to cellulose which is one candidate material for the first extra-cellular precipitate.

Increased specificity of Hot-Spot location can also be achieved by “tethering” the new form of the additional therapeutic agent in a manner similar to that employed to tether the first extra-cellular precipitate.

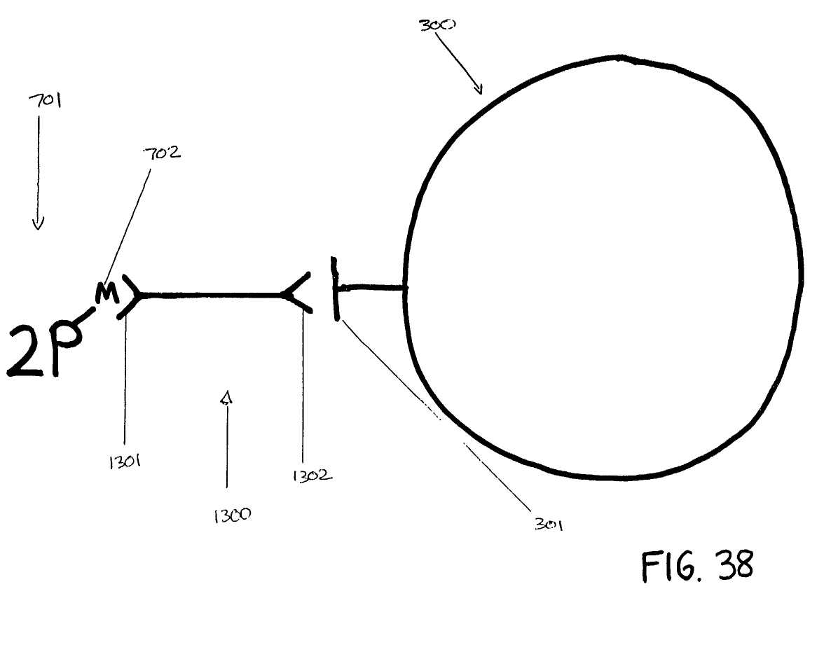

For example, FIG. 38 shows the shows the sixth bispecific reagent 1300 tethering the second-extra-cellular precipitate 701 to the third antigenic receptor 301 of the second target cancer cells 300. The sixth bispecific reagent 1300 being comprised of two moieties, the first moiety 1301 being a targeting agent with an affinity for the additional antigenic epitope 702 of the second extra-cellular precipitate 701. The second moiety 1302 of the sixth bispecific reagent 1300 being capable of binding to the third antigenic receptor 301 of the second target cancer cells 300. The sixth bispecific reagent thereby tethering the second extra-cellular precipitate 701 and retaining it in the extra-cellular fluid of the cancer.

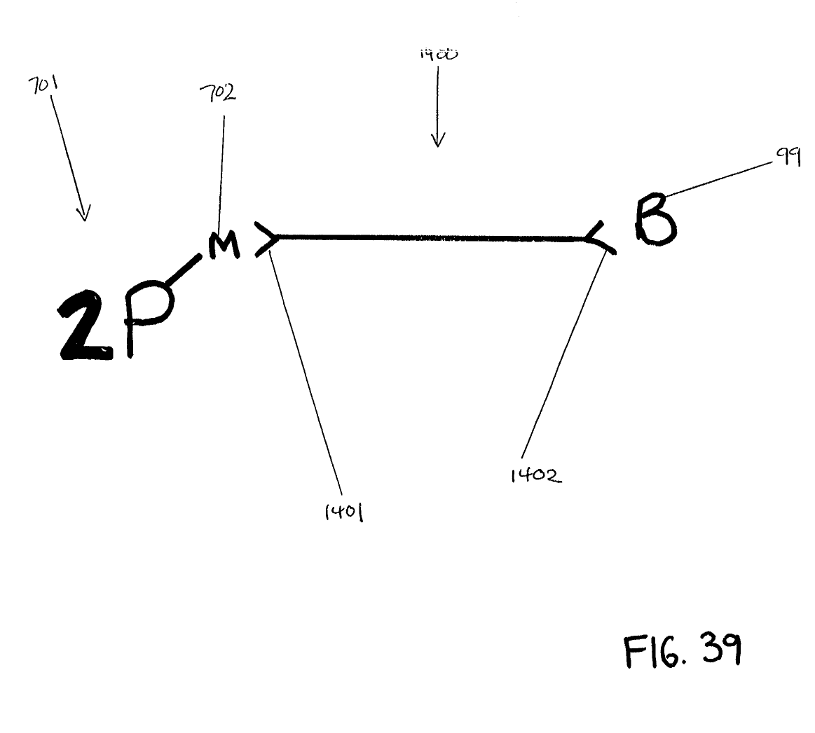

FIG. 39 shows the seventh bispecific reagent 1400 tethering the second extra-cellular precipitate 700 to the antigenic epitopes of the cancer-altered extra-cellular matrix 99. The seventh bispecific reagent 1400 being comprised of two moieties, the first moiety 1401 being a targeting agent with an affinity for the additional antigenic epitope 702 of the second extra-cellular precipitate 701. The second moiety 1402 of the seventh bispecific reagent being capable of binding to the antigenic epitopes of the cancer-altered extra-cellular matrix 99. The seventh bispecific reagent thereby tethering the second extra-cellular precipitate 701 and retaining it in the extra-cellular fluid of the cancer.

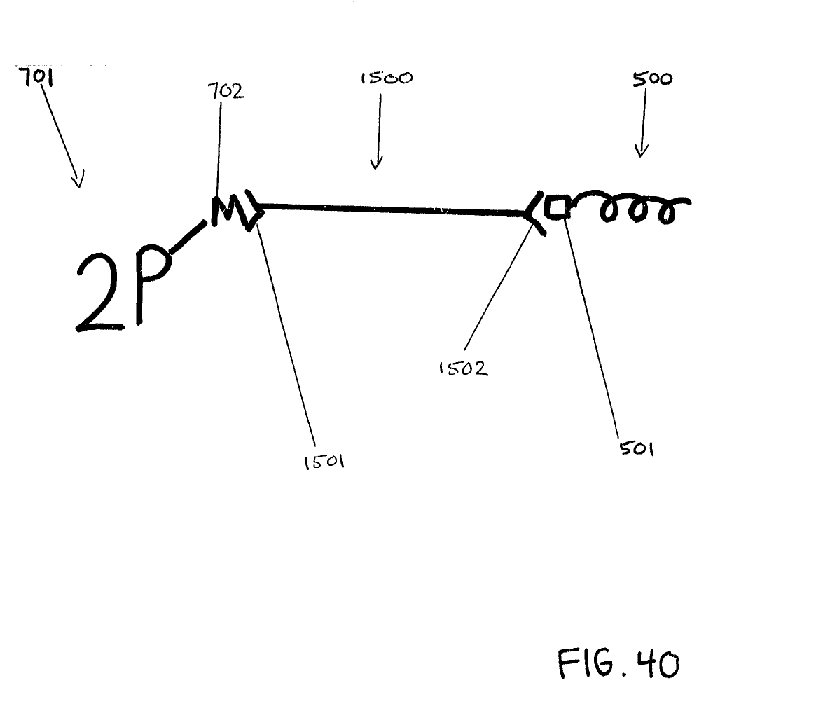

FIG. 40 shows the eighth bispecific reagent 1500 tethering the second extra-cellular precipitate 701 to the antigenic epitopes of the relocated natural intra-cellular material 401. The eighth bispecific reagent 1500 being comprised of two moieties, the first moiety 1501 being a targeting agent with an affinity for the additional antigenic epitope 702 of the second extra-cellular precipitate 701. The second moiety 1502 of the eighth bispecific reagent being capable of binding to the antigenic epitopes of the relocated natural intra-cellular material 501. The eighth bispecific reagent thereby tethering the second extra-cellular precipitate 701 and retaining it in the extra-cellular fluid of the cancer.

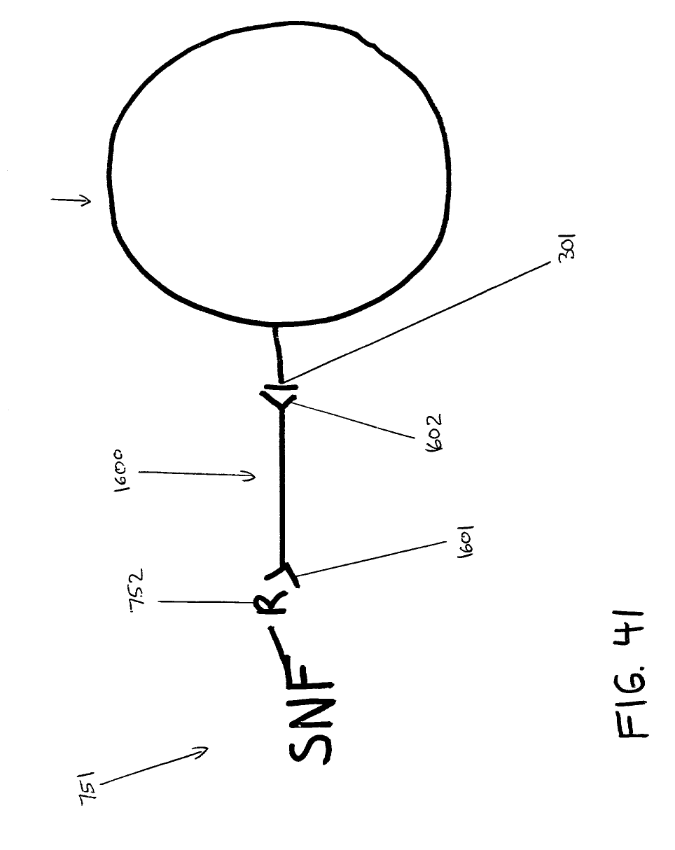

For example, FIG. 41 shows the shows the ninth bispecific reagent 1600 tethering the soluble new form of the third therapeutic agent 751 to the third antigenic receptor 301 of the second target cancer cells 300. The ninth bispecific reagent 1600 being comprised of two moieties, the first moiety 1601 being a targeting agent with an affinity for the additional antigenic epitope 752 of the new form of the third therapeutic agent 751. The second moiety 1602 of the ninth bispecific reagent 1600 being capable of binding to the third antigenic receptor 301 of the second target cancer cells 300. The ninth bispecific reagent thereby tethering the new form of the third therapeutic agent 751 and retaining it in the extra-cellular fluid of the cancer.

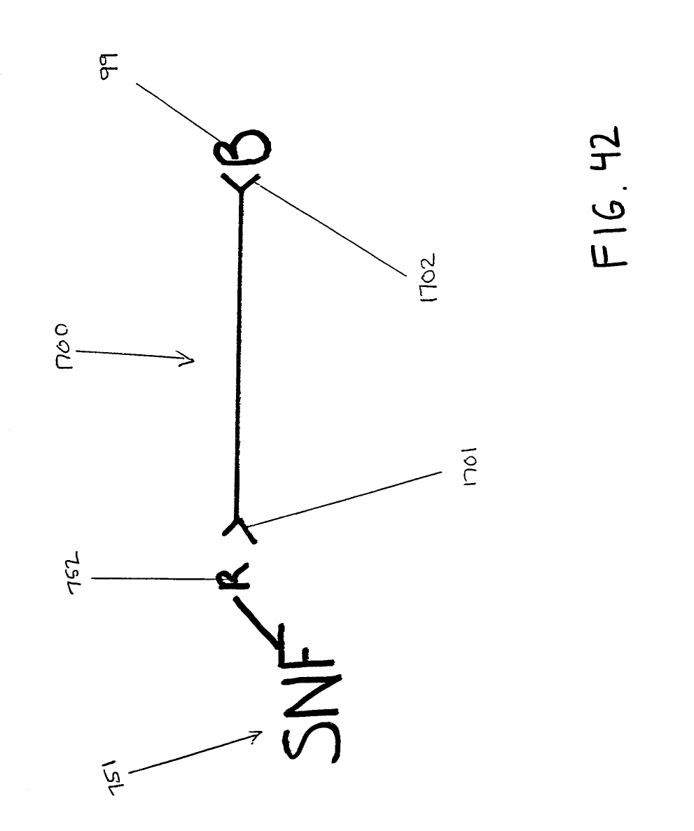

FIG. 42 shows the tenth bispecific reagent 1700 tethering the new form of the third therapeutic agent to the antigenic epitopes of the cancer-altered extra-cellular matrix 99. The tenth bispecific reagent 1700 being comprised of two moieties, the first moiety 1701 being a targeting agent with an affinity for the additional antigenic epitope 752 of the new form of the third therapeutic agent 751. The second moiety 1752 of the tenth bispecific reagent being capable of binding to the antigenic epitopes of the cancer-altered extra-cellular matrix 99. The tenth bispecific reagent thereby tethering the new form of the third therapeutic agent 751 and retaining it in the extra-cellular fluid of the cancer.

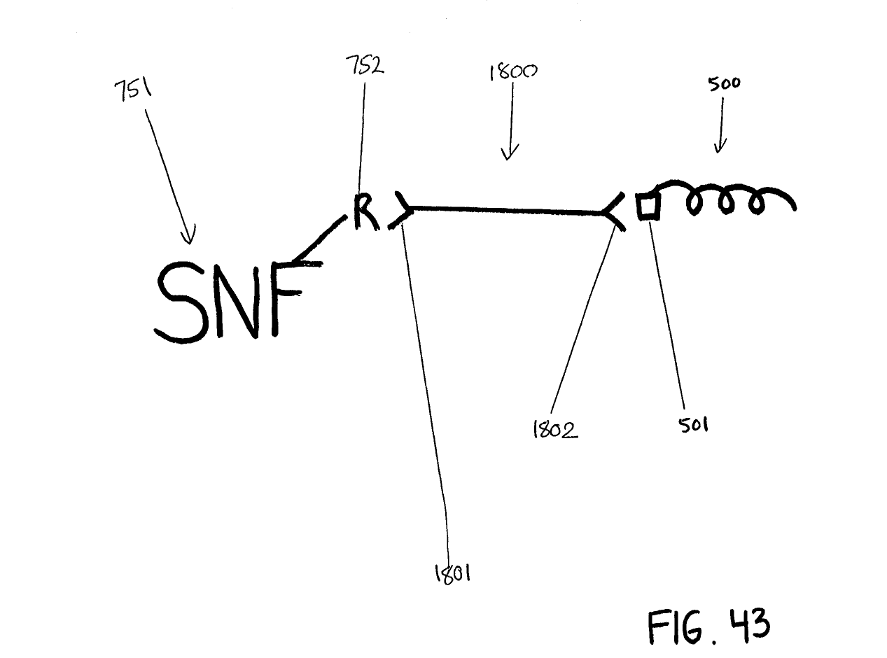

FIG. 43 shows the eleventh bispecific reagent 1800 tethering the new form of the third therapeutic agent 751 to the antigenic epitopes of the relocated natural intra-cellular material 501. The eleventh bispecific reagent 1800 being comprised of two moieties, the first moiety 1801 being a targeting agent with an affinity for the additional antigenic epitope 752 of the new form of the third therapeutic agent 751. The second moiety 1802 of the eleventh bispecific reagent being capable of binding to the antigenic epitopes of the relocated natural intra-cellular material 501. The eleventh bispecific reagent thereby tethering the new form of the third therapeutic agent 751 and retaining it in the extra-cellular fluid of the cancer.

The effectiveness of the fifth, eighth, and eleventh bispecific reagents to tether the first extra-cellular precipitate, the second extra-cellular precipitate, and the new form of the third therapeutic agent respectively depends on the presence in the cancer region of the natural intra-cellular material of cells, in particular the presence of DNA, histone, and complexes of DNA-histone.

In order to increase the effectiveness of the ability of the fifth, eighth, and eleventh bispecific reagents to tether the first extra-cellular precipitate, the second extra-cellular precipitate, and the new form of the third therapeutic agent respectively, the present invention provides the additional step of administering to the living host a cell killing process capable of selectively killing cells in the cancer region and thereby increasing the amount of DNA, histone, and complexes of DNA-histone that will be present in the cancer region.

Theory and data support the view that the low dose selective killing of these super-sensitive cells can be readily achievable. In fact, the frequent presence of dead cells in histological preparations of cancer tissue suggests that some tumor cells are so super-sensitive that they have been killed by various natural host factors which operate at the physiological low levels which prevail in the tumor-bearing subject. It is even possible that in some tumors, the natural host killing is so frequent that no external agent need be administered to achieve the necessary selective killing of the super-sensitive fraction. The presence of super-sensitive cancer cells, reflecting as it does the universal heterogeneity and genetic instability of cancer cell populations, may be the most common, the most specific, and with respect to the method of the invention, the most exploitable characteristic of any cancer. The low dose selective killing of these super-sensitive cancer cells can be considered to be analogous to, or an enhancement of the natural, continuous, and selective killing of some cancer cells by the defense system of the body.

As discussed earlier, the on-going natural killing of cancer cells by the natural immune system of the body may be sufficient to provide the intra-cellular material necessary for tethering via the fifth, eighth, and eleventh bispecific reagents.

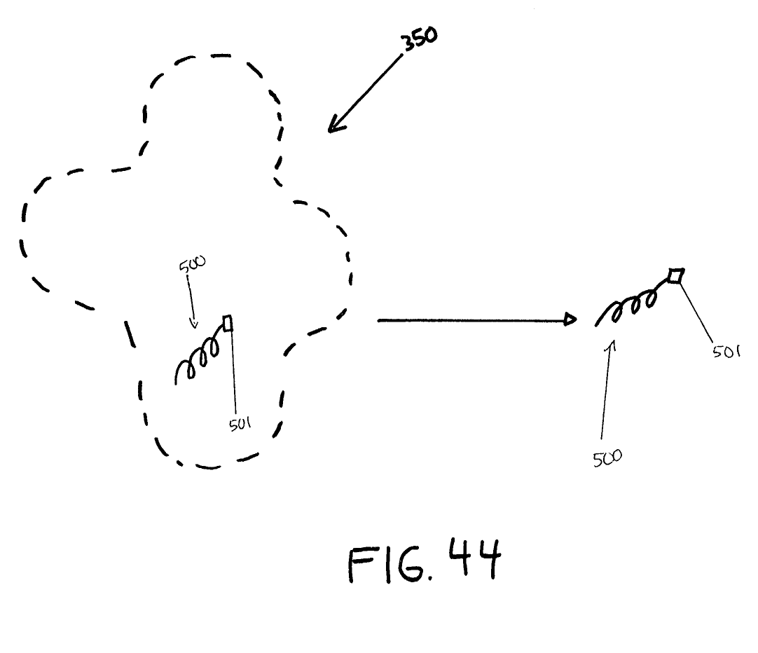

As shown in FIG. 44, according to the method of the present invention is the step of administering to the living host the cell-killing process capable of killing the third target cancer cells 350 thereby causing the natural intracellular material 500 having antigenic epitopes 501 to be relocated into the extra-cellular fluid adjacent to the first target cancer cells and the first target normal cells.

In accordance with the present invention, selective killing of a fraction of cancer cells is achieved by exploiting the heterogeneity of the tumor cell population. The universal and wide heterogeneity which is found in tumor cell populations is well known. It is expressed in every parameter which has been measured in cells, including a heterogeneity in their sensitivity or resistance to being killed by an untoward environment. This untoward environment includes all current agents which are used in an attempt to kill cancer cells, as well as the environment created by the natural system of the living host. As a result of this heterogeneity, it is likely that there will always be some cancer cells which are super-resistant to being killed prior to the administration of any of the current therapeutic agents; however, heterogeneity of the cancer cells expresses itself, not only by the presence of super-resistant cells, but also by the presence of cells which are super-sensitive to being killed. These cancer cells have so many gene errors that they are killed by low doses of the current therapeutic agents or by other cell killing processes. Normal cells do not have these gene errors so that few normal cells (if any) are super-sensitive. Therefore, few normal cells are killed by the very low doses of agents which kill super-sensitive cancer cells.

Current research and therapy regimens ignores the presence of these super-sensitive cancer cells because these cells are of no scientific interest or practical value in the context of current therapies. In contrast, the approach of the present invention exploits the presence of super-sensitive cancer cells and kills them selectively by administering the cell killing process. The cell killing process may include the administration of low doses of anti-cancer agents which are currently available, and preferably which cause lysis of cell membranes.

Death of cells eliminates the normal permeability restrictions to molecules that are characteristic of intact cells and enables molecules like trypan blue and antibodies to gain access to intracellular material. For example, labeled anti-myosin antibody can bind to cardiac myosin when the cardiac cells have been killed (Khaw et al., 1987, J. Nuclear Med., 28, 1671-1678), and anti-histone-DNA antibody can bind to histone-DNA complex when cancer (or other) cells are killed (Epstein et al. 1988, Cancer Research, 48, 5842-5848).

The cell killing process capable of selectively killing cells which have a high sensitivity to being killed by, the natural system of the living host, and having a high sensitivity to being killed by the cell killing process. The cell killing relocates the natural intra-cellular material of cells, including DNA, histone, and complexes of DNA-histone, to the extra-cellular fluid of the cancer, thereby making it available for tethering by the fifth, eighth, and eleventh bispecific reagents.

The cell killing process including at least one of the administration any one of numerous anti-cancer cytotoxic drugs or cellular agents at a low dosage so that only cells with the charactersue of having a high sensitivity to being killed by the cell killing process would be killed. The cell killing process also includes non-toxic agents, such as hormones or anti-hormones, or a procedure, such as orchidectomy, which leads to an alteration in the hormonal status of the living host and causes a cell killing process called apoptosis which is directed against cells of a particular cell lineage which are sensitive to the hormonal status of the living host. For example, orchidectomy and/or the administration of anti-androgens causes the apoptotic killing of a large number of normal prostate cells and a variable number of prostatic cancer cells. Regardless of which cell killing process is employed, the cell killing process is capable of selectively killing at least cells with the characteristic of having a high sensitivity to being killed by the cell killing process.

What is claimed is:

1. A method for treating a heterogeneous population of cancer cells in a living host by at least one of a first therapeutic agent and an additional therapeutic agent, the living host being including normal cells growing in a normal extra-cellular matrix having at least collagen and fibronectin, the heterogeneous population of cancer cells growing in a cancer-altered extra-cellular matrix having at least cancer-altered antigenic epitopes, the heterogeneous population of cancer cells endogenously making and containing products including at least sulphated glycosaminoglycans, natural intra-cellular enzymes in the lysosomes, and natural intra-cellular material including DNA, histone, and complexes of DNA-histone, the DNA, histone, and complexes of DNA-histone having antigenic epitopes, the heterogeneous population of cancer cells including at least four sub-populations of cancer cells:
the first sub-population of cancer cells being first target cancer cells each having a first antigenic receptor which is substantially specific to a cancer cell and which is capable of binding a first targeting agent, the first antigenic receptor being incapable of endocytosis when the first targeting agent binds to the first antigenic receptor;
a second sub-population of cancer cells being the second target cancer cells each having a second antigenic receptor which is substantially specific to a cancer cell and which is incapable of endocytosis;
a third sub-population of cancer cells being the third target cancer cells each having a high sensitivity to being killed by the natural system of the living host and a high sensitivity to being killed by the natural system of the living host;
a fourth sub-population of cancer cells being non-target cancer cells which are the remainder of the heterogeneous population of the cancer cells; and
the normal cells of the living host in addition endogenously making and containing products including at least sulphated glycosaminoglycans, natural intra-cellular enzymes in the lysosomes, and natural intra-cellular material including DNA, histone, and complexes of DNA-histone, the DNA, histone and complexes of DNA-histone having antigenic epitopes, the normal cells including at least two sub-populations of normal cells:
the first sub-population of normal cells being the first target normal cells having the first antigenic receptor which is capable of binding the first targeting agent, the first antigenic receptor being incapable of endocytosis when the first targeting agent binds to the first antigenic receptor;
a second sub-population of normal cells being non-target normal cells which are the remainder of the normal cells;
the method comprising the steps of:
introducing into the living host a first bispecific reagent having two moieties, a first moiety which is a non-mammalian enzyme moiety being a first enzyme moiety, the first bispecific reagent further having a second moiety including a targeting agent moiety which has a substantial affinity for the first antigenic receptor of the first target cancer cells and the first target normal cells;
permitting the first bispecific reagent to bind to the first antigenic receptor of the first target cancer cells and of the first target normal cells, the first bispecific reagent being received and bound at the first antigenic receptor of the first target cancer cells and of the first target normal cells, the first bispecific reagent thereby being retained in the extra-cellular fluid for an extended period of time which enables the first enzyme moiety to convert a substantial amount of the first therapeutic agent in the extra-cellular fluid into an insoluble non-digestible precipitate which is a first extra-cellular precipitate, the first extra-cellular precipitate being capable of remaining in the extra-cellular fluid adjacent to the first bispecific reagent for an extended period of time;
administering to the living host the first therapeutic agent which is a soluble precipitable material and which is converted by the first enzyme moiety of the first bispecific reagent into the first extra-cellular precipitate, the first extra-cellular precipitate having at least one of a first antigenic epitope being an epitope which is an integral part of the structure of the first extra-cellular precipitate, a second antigenic epitope, and a neo-antigenic third epitope, the first extra-cellular precipitate forming in the extra-cellular fluid adjacent to the first bispecific reagent and being capable of remaining in the extra-cellular fluid adjacent to the first bispecific reagent for an extended period of time;
continuing the introducing of the first therapeutic agent into the living host to increase the amount of the first extra-cellular precipitate forming in the extra-cellular fluid, the continued administration of the first therapeutic agent thereby causing an accumulation of first extra-cellular precipitate to form in the extra-cellular fluid, the accumulation of the first extra-cellular precipitate thereby having a plurality of antigenic epitopes which is proportional to the amount of accumulation;
additionally introducing to the living host a second bispecific reagent having two moieties, a first moiety being a non-mammalian enzyme moiety which is a second enzyme moiety including a targeting agent moiety having a substantial affinity for at least one of the first antigenic epitope, the second antigenic epitope, and the neo-antigenic third epitope of the first extra-cellular precipitate;
further permitting the second bispecific reagent to bind to at least one of the first antigenic epitope, the second antigenic epitope, and the neo-antigenic third epitope of the first extra-cellular precipitate, the second bispecific reagent being received and bound at the first extra-cellular precipitate which is retained in the extra-cellular fluid for an extended period of time, thereby enabling the second enzyme moiety to convert a substantial amount of an additional therapeutic agent into a new form capable of remaining in the extra-cellular fluid adjacent to the first extra-cellular precipitate for an extended period of time which is sufficient to kill non-selectively all cells adjacent to the first extra-cellular precipitate; and
additionally administering to the living host the additional therapeutic agent which is a soluble radioactive toxic agent to be converted by the second enzyme moiety into the new form cable of remaining in the extra-cellular fluid adjacent to the first extra-cellular precipitate for an extended period of time which is sufficient to kill non-selectively all cells adjacent to the first extra-cellular precipitate.

2. A method in accordance with claim 1 in which the first enzyme moiety is beta lactamase.

3. A method in accordance with claim 1 in which the first enzyme moiety is a penicillinase.

4. A method in accordance with claim 1 in which the first enzyme moiety is a glycosidase.

5. A method in accordance with claim 1 in which the first therapeutic agent is a soluble agent and is an organic chemical comprising at least one of peptides, including opio-melanins, of carbohydrates including cellulose, chitosan, and chitin, of proteoglycans, of synthetic polymers, and of indoxyl compounds having molecular positions 1-7.

6. A method in accordance with claim 1 in which the first therapeutic agent is inherently cell impermeant.

7. A method in accordance with claim 1 in which a cell-impermeant chemical is attached to the first therapeutic agent the cell-impermeant chemical causing the additional therapeutic agent to be cell impermeant.

8. A method in accordance with claim 7 in which the cell-impermeant chemical includes one of thiol, anionic materials, and materials having a molecular weight greater than 1000 daltons.

9. A method in accordance with claim 1 in which the first therapeutic agent is inherently a soluble molecule which is converted by the first enzyme moiety of the first bispecific reagent into a new form which is insoluble and forms the first extra-cellular precipitate.

10. A method in accordance with claim 1 in which the first therapeutic agent is inherently a soluble molecule which is converted by the first enzyme moiety of the first bispecific reagent into a new form which is insoluble and forms the first extra-cellular precipitate, the first extra-cellular precipitate having a neo-antigenic epitope not present on the first therapeutic agent from which the first extra-cellular precipitate was formed.

11. A method in accordance with claim 1 in which the first therapeutic agent is converted by the first enzyme moiety of the first bispecific reagent into a soluble intermediate molecule, the soluble intermediate molecule being naturally converted in the extra-cellular fluid into a molecule which is insoluble and forms the first extra-cellular precipitate.

12. A method in accordance with claim 1 in which the first therapeutic agent is rapidly oxidized, the oxidized soluble intermediate molecule spontaneously being dimerized and thereby making a new molecule which is insoluble and forms the first extra-cellular precipitate, the first extra-cellular precipitate having a neo-antigenic epitope not present on the first therapeutic agent from which it was formed.

13. A method in accordance with claim 5 in which the indoxyl compounds include at least one of indoxyl-penicillin, indoxyl-cephalosporin, indoxyl-glycosides and the like which when attached to position 3 of the indoxyl compounds are cleavable by the first enzyme moiety of the bispecific reagent, the material remaining after cleaving at position 3 being a soluble reactive intermediate molecule which oxidizes and dimerizes to make a new insoluble molecule which is insoluble and forms the first extra-cellular precipitate.

14. A method in accordance with claim 5 in which each of the indoxyl compounds includes a substance when attached to at least one of positions 4, 5, 6, and 7 of the indoxyl compound alters the characteristics of the indoxyl compounds and the first extra-cellular precipitate.

15. A method in accordance with claim 5 in which each of the indoxyl compounds includes phenyl compounds attached at position 5 of the indoxyl compound to alter the characteristics of the indoxyl compounds and the first extra-cellular precipitate.

16. A method in accordance with claim 5 in which each of the indoxyl compounds includes benzyloxy compounds and all the derivatives of benzyloxy attached at position 5 of the indoxyl compounds to alter the characteristics of the indoxyl compounds and of the first extra-cellular precipitate.

17. A method in accordance with claim 5 in which each of the indoxyl compounds includes 5,5-bi-indoxyls attached at position 5 of the indoxyl compounds to alter the characteristics of the indoxyl compounds and of the first extra-cellular precipitate.

18. A method in accordance with claim 1 in which the first therapeutic agent has a soluble moiety and an insoluble moiety, the soluble moiety having a solubilizing effect on the insoluble moiety and being cleaved by the first enzyme moiety of the first bispecific reagent from the insoluble moiety, the solubilizing effect of the soluble moiety being thereby dissipated and the remaining material, having been converted into a new form and being insoluble, spontaneously precipitates, and forms the first extra-cellular precipitate.

19. A method in accordance with claim 1 in which the second enzyme moiety is beta lactamase.

20. A method in accordance with claim 1 in which the second enzyme moiety is a penicillinase.

21. A method in accordance with claim 1 in which the second enzyme moiety is a glycosidase.

22. A method in accordance with claim 1 in which the second enzyme moiety is chondroitinase ABC.

23. A method in accordance with claim 1 in which the targeting agent moiety of the second bispecific reagent has a substantial affinity for the first antigenic epitope of the first extra-cellular precipitate.

24. A method in accordance with claim 1 in which the targeting agent moiety of the second bispecific reagent has a substantial affinity for the second antigenic epitope of the first extra-cellular precpitate.

25. A method in accordance to claim 1 in which the targeting agent moiety of the second bispecific reagent has a substantial affinity for the neo-antigenic third epitope of the first extra-cellular precipitate.

26. A method in accordance with claim 1 in which the additional therapeutic agent is a soluble radioactive toxic agent and is an organic chemical comprising at least one of peptides, including opio-melanins, of carbohydrates including cellulose, chitosan, and chitin, of proteoglycans, of synthetic polymers, and of indoxyl compounds having molecular positions 1-7.

27. A method in accordance with claim 1 in which the additional therapeutic agent is inherently cell impermeant.

28. A method in accordance with claim 1 in which a cell-impermeant chemical is attached to the additional therapeutic agent the cell-impermeant chemical causing the additional therapeutic agent to be cell impermeant.

29. A method in accordance with claim 28 in which the cell-impermeant chemical includes one of thiol, anionic materials, and materials having a molecular weight greater than 1000 daltons.

30. A method in accordance with claim 1 in which the additional therapeutic agent being the second therapeutic agent is inherently a soluble molecule which is converted by the second enzyme moiety of the second bispecific reagent into a new form which is insoluble and forms the second extra-cellular precipitate.

31. A method in accordance with claim 1 in which the additional therapeutic agent being the second therapeutic agent is inherently a soluble molecule which is converted by the second enzyme moiety of the second bispecific reagent into a new form which is insoluble and forms the second extra-cellular precipitate, the second extra-cellular precipitate having a neo-antigenic epitope not present on the second therapeutic agent from which the second extra-cellular precipitate was formed.

32. A method in accordance with claim 1 in which the additional therapeutic agent being the second therapeutic agent is converted by the second enzyme moiety of the second bispecific reagent into a soluble intermediate molecule, the soluble intermediate molecule being naturally converted in the extra-cellular fluid into a molecule which is insoluble and forms the second extra-cellular precipitate.

33. A method in accordance with claim 1 in which the additional therapeutic agent being the second therapeutic agent is rapidly oxidized, the oxidized soluble intermediate molecule spontaneously being dimerized and thereby making a new molecule which is insoluble and forms the second extra-cellular precipitate, the second extra-cellular precipitate having a neo-antigenic epitope not present on the second therapeutic agent from which it was formed.

34. A method in accordance with claim 26 in which the indoxyl compounds include at least one of indoxyl-penicillin, indoxyl-cephalosporin, indoxyl-glycosides and the like which when attached to position 3 of the indoxyl compounds are cleavable by the second enzyme moiety of the second bispecific reagent, the material remaining after cleaving at position 3 being a soluble reactive intermediate molecule which oxidizes and dimerizes to make a new insoluble molecule which is insoluble and forms the second extra-cellular precipitate.

35. A method in accordance with claim 26 in which each of the indoxyl compounds includes a substance when attached to at least one of positions 4, 5, 6, and 7 of the indoxyl compound alters the characteristics of the indoxyl compounds and the second extra-cellular precipitate.

36. A method in accordance with claim 26 in which each of the indoxyl compounds includes phenyl compounds attached at position 5 of the indoxyl compound to alter the characteristics of the indoxyl compounds and the second extra-cellular precipitate.

37. A method in accordance with claim 26 in which each of the indoxyl compounds includes benzyloxy compounds and all the derivatives of benzyloxy attached at position 5 of the indoxyl compounds to alter the characteristics of the indoxyl compounds and of the second extra-cellular precipitate.

38. A method in accordance with claim 26 in which each of the indoxyl compounds includes 5,5-bi-indoxyls attached at position 5 of the indoxyl compounds to alter the characteristics of the indoxyl compounds and of the second extra-cellular precipitate.

39. A method in accordance with claim 1 in which the additional therapeutic agent being the second therapeutic agent has a soluble moiety and an insoluble moiety, the soluble moiety having a solubilizing effect on the insoluble moiety and being cleaved by the second enzyme moiety of the second bispecific reagent from the insoluble moiety, the solubilizing effect of the soluble moiety being thereby dissipated and the remaining material, having been converted into a new form and being insoluble, spontaneously precipitates, and forms the second extra-cellular precipitate.

40. A method in accordance with claim 1 in which the additional therapeutic agent being the third therapeutic agent is converted by the second enzyme moiety of the second bispecific reagent into a new form, the new form being a soluble material having a neo-antigenic epitope not present on the third therapeutic agent from which the new form was created.

41. A method in accordance with claim 40 in which the third therapeutic agent is chondroitin sulphate which is converted by the second enzyme moiety of the second bispecific reagent into a new form, the new form of the third therapeutic agent being a soluble material with a neo-antigenic epitope not present on the chondroitin sulphate from which the new form of the third therapeutic agent was created.

42. The method in accordance with claim 40 and further comprising the step of administering to the living host a precipitating antibody having a specific affinity for the neo-antigenic epitope on the new form of the third therapeutic agent, the precipitating antibody being administered prior to the step of administering the third therapeutic agent and having the ability to bind to the neo-antigenic epitope of the new form of the third therapeutic agent, the binding causing the new form of the third therapeutic agent to form a third extra-cellular precipitate which remains for an extended period of time adjacent to the relocated first extra-cellular precipitate.

43. A method according to claim 1 and further comprising the step of administering to the living host a third bispecific reagent to tether the first extra-cellular precipitate, the third bispecific reagent having two moieties, the first moiety having an affinity for one of the first antigenic epitope, the second antigenic epitope, and the neo-antigenic third epitope of the first extra-cellular precipitate, the second moiety having an affinity for the third antigenic receptor on the second target cancer cells, the third bispecific reagent being administered prior to the administration of the first therapeutic agent and enabling the first extra-cellular precipitate to be retained for an extended period of time adjacent to the third antigenic receptor on the second target cancer cells.

44. A method according to 43 in which the first moiety of the third bispecific reagent has an affinity for the first antigenic epitope of the first extra-cellular precipitate.

45. A method according to 43 in which the first moiety of the third bispecific reagent has an affinity for the second antigenic epitope of the first extra-cellular precipitate.

46. A method according to 43 in which the first moiety of the third bispecific reagent has an affinity for the neo-antigenic third epitope of the first extra-cellular precipitate.

47. A method according to claim 1 and further comprising the step of administering to the living host a fourth bispecific reagent, the fourth bispecific reagent having two moieties, the first moiety having an affinity for one of the first antigenic epitope, the second antigenic epitope, and the neo-antigenic third epitope of the first extra-cellular precipitate, the second moiety having an affinity for the cancer-altered antigenic epitopes on the cancer-altered extra-cellular matrix, the fourth bispecific reagent being administered prior to the administration of the first therapeutic agent and enabling the first extra-cellular precipitate to be retained for an extended period of time adjacent to the cancer-altered antigenic epitopes on the cancer-altered extra-cellular matrix.

48. A method according to 47 in which the first moiety of the fourth bispecific reagent has an affinity for the first antigenic epitope of the first extra-cellular precipitate.

49. A method according to 47 in which the first moiety of the fourth bispecific reagent has an affinity for the second antigenic epitope of the first extra-cellular precipitate.

50. A method according to 47 in which the first moiety of the fourth bispecific reagent has an affinity for the neo-antigenic third epitope of the first extra-cellular precipitate.

51. A method according to claim 1 and further comprising the step of administering to the living host a fifth bispecific reagent, the fifth bispecific reagent having two moieties, the first moiety having an affinity for one of the first antigenic epitope, the second antigenic epitope, and the neo-antigenic third epitope of the first extra-cellular precipitate, the second moiety having an affinity for the antigenic epitopes on the relocated natural intra-cellular material, the fifth bispecific reagent being administered prior to the administration of the first therapeutic agent and enabling the first extra-cellular precipitate to be retained for an extended period of time adjacent to the antigenic epitopes on the relocated natural intra-cellular material.

52. A method according to 51 in which the first moiety of the fifth bispecific reagent has an affinity for the first antigenic epitope of the first extra-cellular precipitate.

53. A method according to 51 in which the first moiety of the fifth bispecific reagent has an affinity for the second antigenic epitope of the first extra-cellular precipitate.

54. A method according to 51 in which the first moiety of the fifth bispecific reagent has an affinity for the neo-antigenic third epitope of the first extra-cellular precipitate.

55. A method according to claim 31 and further comprising the step of administering to the living host a sixth bispecific reagent comprised of a molecule having a substantial affinity for the neo-antigenic epitope of the second extra-cellular precipitate, the sixth bispecific reagent further having a molecule with a substantial affinity for the third antigenic receptor on the second target cancer cells, the sixth bispecific reagent being administered prior to the step of additionally administering the second therapeutic agent and enabling the second extra-cellular precipitate to be retained for an extended period of time adjacent to the third antigenic receptor on the second target cancer cells.

56. A method according to claim 31 and further comprising the step of administering to the living host a seventh bispecific reagent comprised of a molecule having a substantial affinity for the neo-antigenic epitope on the second extra-cellular precipitate, the seventh bispecific reagent further having a molecule with a substantial affinity for the cancer-altered antigenic epitopes on the cancer-altered extra-cellular matrix, the seventh bispecific reagent being administered prior to the step of additionally administering the second therapeutic agent and enabling the second extra-cellular precipitate to be retained for an extended period of time adjacent to the cancer-altered antigenic epitopes on the cancer-altered extra-cellular matrix.

57. A method according to claim 31 and further comprising the step of administering to the living host a eighth bispecific reagent comprised of a molecule having a substantial affinity for the neo-antigenic epitope on the second extra-cellular precipitate, the eighth bispecific reagent further having a molecule with a substantial affinity for the antigenic epitopes on the natural intra-cellular material, the eighth bispecific reagent being administered prior to the step of administering the second therapeutic agent and enabling the second extra-cellular precipitate to be retained for an extended period of time adjacent to the antigenic epitopes on the natural intra-cellular material.

58. A method according to claim 40 and further comprising the step of administering to the living host an ninth bispecific reagent comprised of a molecule having a substantial affinity for the neo-antigenic epitope of the new form of the third therapeutic agent, the ninth bispecific reagent further having a molecule with a substantial affinity for the third antigenic receptor on the second target cancer cells, the ninth bispecific reagent to be administered prior to the step of administering the third therapeutic agent and enabling the new form of the third therapeutic agent to be retained for an extended period of time adjacent to the third antigenic receptor on the second target cancer cells.

59. A method according to claim 40 and further comprising the step of administering to the living host a tenth bispecific reagent comprised of a molecule having a substantial affinity for the neo-antigenic epitope on the new form of the third therapeutic agent, the tenth bispecific reagent further having a molecule with a substantial affinity for the cancer-altered antigenic epitopes on the cancer-altered extra-cellular matrix, the tenth bispecific reagent being administered prior to the step of administering the third therapeutic agent and enabling the new form of the third therapeutic agent to be retained for an extended period of time adjacent to the cancer-altered antigenic epitopes on the cancer-altered extra-cellular matrix.

60. A method according to claim 40 and further comprising the step of administering to the living host a eleventh bispecific reagent comprised of a molecule having a substantial affinity for the neo-antigenic epitope on the new form of the third therapeutic agent, the eleventh bispecific reagent further having a molecule with a substantial affinity for the antigenic epitopes on the natural intra-cellular material, the eleventh bispecific reagent being administered prior to the step of administering the third therapeutic agent and enabling the new form of the third therapeutic agent to be retained for an extended period of time adjacent to the antigenic epitopes on the natural intra-cellular material.

61. A method in accordance with claim 17 in which two indoxyl compounds are attached via a spacer molecule.

62. A method in accordance with claim 38 in which two indoxyl compounds are attached via a spacer molecule.

63. A method in accordance with claim 1 in which the first therapeutic agent is radio-labeled.

64. A method in accordance with claim 1 and further comprising the step of administering a cell killing process capable of selectively killing cancer cells that are super-sensitive including at least the third target cancer cells, the killing of the cells thereby causing the natural intra-cellular contents, including DNA, histone, and complexes of DNA-histone to be released into the extra-cellular fluid.

65. A method according to 64 in which the cell killing process includes the administering of at least a cytotoxic agent capable of selectively killing cells.

66. A method in accordance with claim 64 in which the administering of the cell killing process includes the administering of at least a non-cytotoxic agent capable of selectively killing cells.

67. A method in accordance with claim 1 in which the administering of cell killing process includes the administering of a procedure which alters the hormonal status and which is capable of selectively killing cells.

68. A method in accordance with claim 1 in which the administering of the cell killing process includes a cell killing process which is capable of inducing lysis.

69. A first therapeutic agent being a soluble precipitable material which is adapted to be disposed adjacent to a first target cancer cell, a first bispecific reagent having a first enzyme moiety being a non-mammalian enzyme moiety being bound to the first target cancer cell, the first therapeutic agent being adapted to be converted in the extra-cellular fluid adjacent to the first bispecific reagent, into an insoluble and non-digestible precipitate which is a first extra-cellular precipitate by the action of the first enzyme moiety of the first bispecific reagent bound to the first target cancer cell, the first therapeutic agent comprising at least one organic chemical of at least one of peptides, including opio-melanins, of carbohydrates including cellulose, chitosan, and chitin, of proteoglycans, of synthetic polymers, and of indoxyl compounds having molecular positions 1-7, the first extra-cellular precipitate having at least one of a first antigenic epitope being an epitope which is an integral part of the structure of the first extra-cellular precipitate, a second antigenic epitope, and a neo-antigenic third epitope, the neo-antigenic third epitope not being present on the first therapeutic agent, the first extra-cellular precipitate remaining in the extra-cellular fluid adjacent to the first bispecific reagent for an extended period of time.

70. A first therapeutic agent in accordance with claim 69 in which the first therapeutic agent is inherently cell impermeant.

71. A first therapeutic agent in accordance with claim 69 in which a cell-impermeant chemical is attached to the first therapeutic agent, the cell-impermeant chemical causing the first therapeutic agent to be cell impermeant.

72. A first therapeutic agent in accordance with claim 71 in which the cell-impermeant chemical includes one of thiol, anionic materials, and materials having a molecular weight greater than 1000 daltons.

73. A first therapeutic agent in accordance with claim 69 which is inherently soluble.

74. A first therapeutic agent in accordance with claim 69 in which the conversion of the first therapeutic agent comprises the conversion of the first therapeutic agent into a soluble intermediate molecule, the soluble intermediate molecule being adapted to be naturally converted in the extra-cellular fluid into the first extra-cellular precipitate.

75. A first therapeutic agent in accordance with claim 74 in which the soluble intermediate molecule is adapted to be rapidly oxidized, the oxidized soluble intermediate molecule being adapted to be spontaneously dimerized and thereby forming the first extra-cellular precipitate.

76. A first therapeutic agent in accordance with claim 69 in which each of the indoxyl compounds include at least one of indoxyl-penicillin, indoxyl-cephalosporin, indoxyl-glycosides and the like which when attached to position 3 of the indoxyl compounds are cleavable by the first enzyme moiety of the first bispecific reagent, the material remaining after cleaving at position 3 being a soluble reactive intermediate molecule which is adapted to be oxidized and dimerized thereby forming the first extra-cellular precipitate.

77. A first therapeutic agent in accordance with claim 69 in which each of the indoxyl compounds includes a substance which when attached to at least one of positions 4, 5, 6, and 7 of the indoxyl compound alters the characteristics of the indoxyl compounds and the first extra-cellular precipitate.

78. A first therapeutic agent in accordance with claim 69 in which each of the indoxyl compounds includes phenyl compounds attached at position 5 of the indoxyl compound to alter the characteristics of the indoxyl compounds and the first extra-cellular precipitate.

79. A first therapeutic agent in accordance with claim 69 in which each of the indoxyl compounds includes benzyloxy compounds and derivatives of benzyloxy attached at position 5 of the indoxyl compounds to alter the characteristics of the indoxyl compounds and of the first extra-cellular precipitate.

80. A first therapeutic agent in accordance with claim 69 in which each of the indoxyl compounds includes 5,5-bi-indoxyls attached at position 5 of the indoxyl compounds to alter the characteristics of the indoxyl compounds and of the first extra-cellular precipitate thereof.

81. A first therapeutic agent in accordance with claim 80 in which two indoxyl compounds are attached via a spacer molecule.

82. A first therapeutic agent in accordance with claim 69 which has a soluble moiety and an insoluble moiety, the soluble moiety having a solubilizing effect on the insoluble moiety and being cleaved by the first enzyme moiety of the first bispecific reagent from the insoluble moiety, the solubilizing effect of the soluble moiety being thereby dissipated and the remaining material being adapted to form the first extra-cellular precipitate.

83. A first therapeutic agent in accordance with claim 69 which is radio-labeled.

84. A bispecific reagent which is adapted to be received and bound at a first antigenic receptor which is a non-endocytosing receptor of a first target cancer cell, the bispecific reagent being comprised of two moieties, the first moiety being a non-mammalian enzyme which is a first enzyme moiety and being adapted to convert a first therapeutic agent into an insoluble non-digestible precipitate which is a first extra-cellular precipitate, the first bispecific reagent further having a second moiety which is a targeting agent moiety adapted to have a substantial affinity for the first antigenic receptor of the first target cancer cell.

85. A bispecific reagent according to 84 in which the first enzyme moiety is beta lactamase.

86. A bispecific reagent according to 84 in which the first enzyme moiety is a penicillinase.

87. A bispecific reagent according to 84 in which the first enzyme moiety is a glycosidase.
